(19) United States
(12) Patent Application Publication
(10) Pub. No.: US 2024/0238374 A1
(43) Pub. Date: Jul. 18, 2024

(54) METHODS AND COMPOSITIONS FOR INDUCING BROWN ADIPOGENESIS

(71) Applicant: ENERGESIS PHARMACEUTICALS, INC., CAMBRIDGE, MA (US)

(72) Inventors: OLIVIER D. BOSS, CAMBRIDGE, MA (US); BRIAN FREEMAN, CAMBRIDGE, MA (US)

(21) Appl. No.: 18/561,769

(22) PCT Filed: May 20, 2022
(86) PCT No.: PCT/US2022/030299

§ 371 Date: Nov. 17, 2023

Related U.S. Application Data
(60) Provisional application No. 63/191,271, filed on May 20, 2021.

(51) Int. Cl. A61K 38/18; A61K 31/195; A61K 31/421; A61K 45/06; A61P 3/04; A61P 3/10

(52) U.S. Cl. CPC A61K 38/1825; A61K 31/195; A61K 31/421; A61K 45/06; A61P 3/04; A61P 3/10

(57) ABSTRACT

This disclosure features compositions and methods for the treatment of metabolic disorders such as diabetes and obesity.

### CROSS-REFERENCE TO RELATED APPLICATION

This application claims the benefit of U.S. Ser. No. 63/191,271, filed May 20, 2021, the disclosure of which is hereby incorporated by reference in its entirety, including all figures, tables and amino acid or nucleic acid sequences.

### STATEMENT REGARDING SEQUENCE LISTING

The Sequence Listing for this application is labeled “Seq-List.txt” which was created on May 20, 2022 and is 24513 bytes. The entire content of the sequence listing is incorporated herein by reference in its entirety.

### TECHNICAL FIELD

The present disclosure relates to compositions and methods related to enhancing brown adipocytes, and/or brown adipocyte mass, in conditions such as type 2 diabetes, obesity, insulin-resistance, and dyslipidemia and which results in body weight loss and improves other parameters of metabolic health such as blood glucose and insulin through the recruitment of brown adipocyte stem/progenitor cells to increase the mass of brown adipose tissue (BAT) and increase energy expenditure or metabolic rate with no significant effects on food intake.

### BACKGROUND

The epidemic of obesity is closely associated with increases in the prevalence of diabetes, hypertension, coronary heart disease, cancer and other disorders. The role of white adipose tissue is to store lipids, and it is associated with obesity. The role of brown adipose tissue (“BAT”) is effectively the opposite. It is specialized in lipid combustion and the dissipation of energy as heat. Indeed, the brown adipocyte contains numerous mitochondria (in which cellular combustion occurs) and uniquely expresses uncoupling protein-1 (“UCP1”). UCP1 acts as an uncoupler of oxidative phosphorylation, resulting in dissipation of energy as heat. The sympathetic nervous system stimulates mitochondriogenesis and UCP1 expression and activity. BAT-associated thermogenesis in rodents is increased upon exposure to low temperature (e.g., preventing hypothermia) or as a result of overeating, burning excess absorbed fat and preventing weight gain. BAT, by modifying susceptibility to weight gain and by consuming large amounts of glucose, also improves insulin sensitivity. It therefore plays an important role in the maintenance of body temperature, energy balance and glucose metabolism.

Experiments with transgenic animals support the potential anti-obesity properties of BAT. For example, the genetic ablation of BAT has been reported to cause obesity, while genetic increase in the amount and/or function of BAT (and/or UCP1 expression) reportedly promotes a lean and healthy phenotype. Specifically, mice with a higher amount of BAT gain less weight and are more insulin-sensitive than control mice. Recently, ectopic BAT depots were evidenced in the mouse muscle, which have been shown to provide a genetic mechanism of protection from weight gain and metabolic syndrome.

### SUMMARY

The present disclosure provides compositions for the treatment of metabolic disease, including obesity, excess body fat, overweight, diabetes, hyperglycemia, insulin resistance, hyperlipidemia, and other conditions in a patient or animal. The methods disclosed herein utilize a combination of compounds that affect energy expenditure, for example enhance energy expenditure, in subjects or animals treated with the compounds.

This disclosure demonstrates that human Fibroblast Growth Factor-7 (hFGF7) and analogs thereof induce body weight loss and improve other parameters of metabolic health such as blood glucose and insulin through the recruitment of brown adipocyte stem/progenitor cells to increase the mass of BAT and increases energy expenditure or metabolic rate with no significant effects on food intake. Surprisingly, applicants found that the effects were enhanced when a combination of agents was used. For example, when bezafibrate and oxaprozin were co-administered with the GLP-1 receptor agonist semaglutide, the effects on body weight, and several other parameters of metabolic health were more than additive. These findings would not be expected by those skilled in the art. In addition, it was found that the effects of hFGF7, when used in combination with GLP-1 receptor agonists were much greater than expected. For example, when hFGF7 was co-administered with the GLP-1 receptor agonist semaglutide, the effects on body weight were more than additive.

### BRIEF DESCRIPTION OF THE DRAWINGS

FIG. 1 shows the effects of the combination of bezafibrate (60 mg/kg BW by oral gavage once a day)+oxaprozin (50 mg/kg BW by oral gavage once a day, also known as EGS2632) and semaglutide (0.012 mg/kg BW by intraperitoneal injection every 3 days) on body weight in Diet-Induce Obese (DIO) mice. Bezafibrate+oxaprozin with semaglutide synergistically lowered body weight.

FIG. 2 shows the effects of the combination of bezafibrate (60 mg/kg BW by oral gavage once a day)+oxaprozin (50 mg/kg BW by oral gavage once a day) and semaglutide (0.012 mg/kg BW by intraperitoneal injection every 3 days) on body fat (measured by magnetic resonance imaging, MRI) in DIO mice (body fat in grams). Bezafibrate+oxaprozin with semaglutide synergistically lowered body fat mass.

FIG. 3 shows the effects of the combination of bezafibrate (60 mg/kg BW by oral gavage once a day)+oxaprozin (50 mg/kg BW by oral gavage once a day) and semaglutide (0.012 mg/kg BW by intraperitoneal injection every 3 days) on liver fat (liver triglycerides (%)/liver weight) in DIO mice. Bezafibrate+oxaprozin with semaglutide synergistically lowered liver fat.

FIG. 4 shows the effects of the combination of bezafibrate (60 mg/kg BW by oral gavage once a day)+oxaprozin (50 mg/kg BW by oral gavage once a day) and semaglutide (0.012 mg/kg BW by intraperitoneal injection every 3 days) on plasma leptin levels in DIO mice. Leptin is a hormone produced by white adipocytes that serves as a measure of total body fat stores. Bezafibrate+oxaprozin with semaglutide synergistically lowered leptin levels.

FIG. 5 shows the effects of the combination of bezafibrate (60 mg/kg BW by oral gavage once a day)+oxaprozin (50 mg/kg BW by oral gavage once a day) with either semaglutide (0.012 mg/kg BW by intraperitoneal injection every 3 days), exenatide (0.05 mg/kg by intraperitoneal injection every day), or lixisenatide (0.243 mg/kg by intraperitoneal injection every day), on blood glucose levels in DIO mice. It should be noted that while the DIO mouse is a model of obesity and insulin resistance (prediabetes), mice have only mildly elevated non-fasting and fasting blood glucose.

FIG. 6 shows the effects of the combination of bezafibrate (60 mg/kg BW by oral gavage once a day)+oxaprozin (50 mg/kg BW by oral gavage once a day) with either semaglutide (0.012 mg/kg BW by intraperitoneal injection every 3 days), exenatide (0.05 mg/kg by intraperitoneal injection every day), or lixisenatide (0.243 mg/kg by intraperitoneal injection every day), on plasma insulin levels in DIO mice. Bezafibrate+oxaprozin with semaglutide synergistically lowered insulin (pvalue 3.1e-03); bezafibrate+oxaprozin with exenatide or with lixisenatide showed a trend toward synergy (pvalues of 1.0e-01 and 1.2e-01, respectively).

FIG. 7 shows the effects of the combination of bezafibrate (60 mg/kg BW by oral gavage once a day)+oxaprozin (50 mg/kg BW by oral gavage once a day) and semaglutide (0.012 mg/kg BW by intraperitoneal injection every 3 days) on insulin sensitivity in DIO mice, as determined using the homeostasis model assessment of insulin resistance (HOMA-IR). Bezafibrate+oxaprozin with semaglutide reduced HOMA-IR beyond the reduction achieved with semaglutide alone.

FIGS. 8 and 8A show the effects of the combination of hFGF7 at 1 mg/kg BW or at 0.5 mg/kg BW (by intraperitoneal injection once a day) and semaglutide (0.012 mg/kg BW by intraperitoneal injection every 3 days) on body weight in DIO mice (FIG. 8). FIG. 8A shows body weight at study terminus. Both doses of hFGF7 with semaglutide synergistically lowered body weight.

FIGS. 9 and 9A show the effects of the combination of hFGF7 at 1 mg/kg BW or at 0.5 mg/kg BW (by intraperitoneal injection once a day) and semaglutide (0.012 mg/kg BW by intraperitoneal injection every 3 days) on epididymal white adipose (WATepi) depot weight (an index of body fat mass) in DIO mice. The results are shown as a percentage of body weight (FIG. 9) or in mg (FIG. 9A).

FIG. 10 shows the effects of the combination of hFGF7 at 1 mg/kg BW or at 0.5 mg/kg BW (by intraperitoneal injection once a day) and semaglutide (0.012 mg/kg BW by intraperitoneal injection every 3 days) on plasma leptin levels in DIO mice.

FIG. 11 shows the effects of the combination of hFGF7 at 1 mg/kg BW or at 0.5 mg/kg BW (by intraperitoneal injection once a day) and semaglutide (0.012 mg/kg BW by intraperitoneal injection every 3 days) on blood glucose levels in DIO mice.

FIG. 12 shows the effects of the combination of hFGF7 at 1 mg/kg BW or at 0.5 mg/kg BW (by intraperitoneal injection once a day) and semaglutide (0.012 mg/kg BW by intraperitoneal injection every 3 days) on plasma insulin levels in DIO mice.

FIG. 13 shows the effects of the combination of hFGF7 at 1 mg/kg BW or at 0.5 mg/kg BW (by intraperitoneal injection once a day) and semaglutide (0.012 mg/kg BW by intraperitoneal injection every 3 days) on insulin sensitivity in DIO mice, as determined using the homeostasis model assessment of insulin resistance (HOMA-IR).

Similarly to the combination of EGS2632 with semaglutide or other GLP-1R agonists, the magnitude of the effects of the combination of FGF7 or analogs thereof and semaglutide on body weight could not have been anticipated based on the known effects of these individual agents. In fact, surprisingly, synergistic effects were observed.

FIG. 14 shows the effects of the combination of bezafibrate (60 mg/kg BW by oral gavage once a day)+oxaprozin (50 mg/kg BW by oral gavage once a day) and exenatide (0.05 mg/kg by intraperitoneal injection every day) on body weight in DIO mice. Bezafibrate+oxaprozin with exenatide synergistically lowered body weight.

FIG. 15 shows the effects of the combination of bezafibrate (60 mg/kg BW by oral gavage once a day)+oxaprozin (50 mg/kg BW by oral gavage once a day) and exenatide (0.05 mg/kg by intraperitoneal injection every day) on body fat (measured by magnetic resonance imaging, MRI) in DIO mice (body fat in grams). Bezafibrate+oxaprozin with exenatide synergistically lowered body fat mass.

FIG. 16 shows the effects of the combination of bezafibrate (60 mg/kg BW by oral gavage once a day)+oxaprozin (50 mg/kg BW by oral gavage once a day) and exenatide (0.05 mg/kg by intraperitoneal injection every day) on liver fat (liver triglycerides (%)/liver weight) in DIO mice. Bezafibrate+oxaprozin with exenatide synergistically lowered liver fat.

FIG. 17 shows the effects of the combination of bezafibrate (60 mg/kg BW by oral gavage once a day)+oxaprozin (50 mg/kg BW by oral gavage once a day) and exenatide (0.05 mg/kg by intraperitoneal injection every day) on plasma leptin levels in DIO mice. Leptin is a hormone produced by white adipocytes that serves as a measure of total body fat stores. Bezafibrate+oxaprozin with exenatide reduced leptin levels beyond the reduction achieved with exenatide alone.

FIG. 18 shows the effects of the combination of bezafibrate (60 mg/kg BW by oral gavage once a day)+oxaprozin (50 mg/kg BW by oral gavage once a day) and exenatide (0.05 mg/kg by intraperitoneal injection every day) on insulin sensitivity in DIO mice, as determined using the homeostasis model assessment of insulin resistance (HOMA-IR). Bezafibrate+oxaprozin with exenatide synergistically lowered insulin resistance.

FIG. 19 shows the effects of the combination of bezafibrate (60 mg/kg BW by oral gavage once a day)+oxaprozin (50 mg/kg BW by oral gavage once a day) and lixisenatide (0.243 mg/kg by intraperitoneal injection every day) on body weight in DIO mice. Bezafibrate+oxaprozin with lixisenatide synergistically lowered body weight.

FIG. 20 shows the effects of the combination of bezafibrate (60 mg/kg BW by oral gavage once a day)+oxaprozin (50 mg/kg BW by oral gavage once a day) and lixisenatide (0.243 mg/kg by intraperitoneal injection every day) on body fat (measured by magnetic resonance imaging, MRI) in DIO mice (body fat in grams). Bezafibrate+oxaprozin with lixisenatide synergistically lowered body fat mass.

FIG. 21 shows the effects of the combination of bezafibrate (60 mg/kg BW by oral gavage once a day)+oxaprozin (50 mg/kg BW by oral gavage once a day) and lixisenatide (0.243 mg/kg by intraperitoneal injection every day) on liver fat (liver triglycerides (%)/liver weight) in DIO mice. Bezafibrate+oxaprozin with lixisenatide synergistically lowered liver fat.

FIG. 22 shows the effects of the combination of bezafibrate (60 mg/kg BW by oral gavage once a day)+oxaprozin (50 mg/kg BW by oral gavage once a day) and lixisenatide (0.243 mg/kg by intraperitoneal injection every day) on plasma leptin levels in DIO mice. Leptin is a hormone produced by white adipocytes that serves as a measure of total body fat stores. Bezafibrate+oxaprozin with lixisenatide synergistically lowered leptin levels.

FIG. 23 shows the effects of the combination of bezafibrate (60 mg/kg BW by oral gavage once a day)+oxaprozin (50 mg/kg BW by oral gavage once a day) and lixisenatide (0.243 mg/kg by intraperitoneal injection every day) on insulin sensitivity in DIO mice, as determined using the homeostasis model assessment of insulin resistance (HOMA-IR). Bezafibrate+oxaprozin with lixisenatide reduced insulin resistance beyond the reduction achieved with lixisenatide alone.

FIG. 24 shows the effects of the combination of bezafibrate (60 mg/kg BW by oral gavage once a day)+oxaprozin (50 mg/kg BW by oral gavage once a day) and dulaglutide (0.6 mg/kg by intraperitoneal injection once weekly) on body weight in DIO mice. Bezafibrate+oxaprozin with dulaglutide synergistically lowered body weight.

FIG. 25 shows the effects of the combination of bezafibrate (60 mg/kg BW by oral gavage once a day)+oxaprozin (50 mg/kg BW by oral gavage once a day) and dulaglutide (0.6 mg/kg by intraperitoneal injection once weekly) on body fat (measured by magnetic resonance imaging, MRI) in DIO mice (body fat in grams). Bezafibrate+oxaprozin with dulaglutide synergistically lowered body fat mass.

FIG. 26 shows the effects of the combination of bezafibrate (60 mg/kg BW by oral gavage once a day)+oxaprozin (50 mg/kg BW by oral gavage once a day) and dulaglutide (0.6 mg/kg by intraperitoneal injection once weekly) on liver fat (liver triglycerides (%)/liver weight) in DIO mice. Bezafibrate+oxaprozin with dulaglutide synergistically lowered liver fat.

### DETAILED DESCRIPTION

The present disclosure provides compositions for the treatment of metabolic disease, including obesity, excess body fat, overweight, diabetes, hyperglycemia, insulin resistance, hyperlipidemia, and others conditions in a patient. The methods disclosed herein utilize a combination of compounds that affect energy expenditure, for example enhance energy expenditure, in subjects treated with the compounds.

In addition, it has been found that two different compounds used together can provide synergistic effects on body weight, liver fat, body fat, leptin levels, and/or insulin resistance such that the effect is greater than the effect that can be obtained with the compounds alone. For example, one combination of compounds is bezafibrate and ozaprozin in combination with a Glucagon-Like Peptide-1 (GLP-1) receptor agonist, for example, dulaglutide, semaglutide, exenatide, liraglutide, lixisenatide, albiglutide, tirzepatide, danuglipron (PF-06882961), PF-07081532, or LY3502970. Another combination of compounds is human Fibroblast Growth Factor-7 (hFGF7) or analogs thereof in combination with a Glucagon-Like Peptide-1 (GLP-1) receptor agonist, for example, dulaglutide, semaglutide, exenatide, liraglutide, lixisenatide, albiglutide, tirzepatide, danuglipron (PF-06882961), PF-07081532, or LY3502970. Another combination of compounds that can provide an effect on body weight, liver fat, body fat, leptin levels, and/or insulin resistance is bezafibrate or analogs thereof in combination with a Glucagon-Like Peptide-1 (GLP-1) receptor agonist, for example, dulaglutide, semaglutide, exenatide, liraglutide, lixisenatide, albiglutide, tirzepatide, danuglipron (PF-06882961), PF-07081532, or LY3502970.

Accordingly, in one aspect, the disclosure features methods of treating a subject, e.g., decreasing fat stores or weight in a subject such as a human. The methods include administering to the subject a combination of compounds disclosed herein. In a further aspect, the disclosure features methods of administering a population of compound-activated BAT progenitor cells, wherein said population of compound-activated progenitor cells undergo brown adipogenesis following stimulation with a combination of compounds as disclosed herein.

The methods can optionally include identifying a subject in need of decreasing fat stores or weight. In a further aspect, the disclosure includes methods of enhancing insulin sensitivity in a subject, e.g., a subject that is insulin-resistant. The methods include administering to the subject a compound, or a population of compound-activated BAT progenitor cells, wherein said population of compound-activated BAT progenitor cells undergo brown adipogenesis. The methods can optionally include identifying a subject in need of enhanced insulin sensitivity.

In another aspect, the disclosure features methods of modulating brown adipose tissue function or development, e.g., promoting BAT adipogenesis, in a subject. The methods include administering to the subject a combination of compounds or a population of compound-activated BAT progenitor cells, wherein said population of compound-activated progenitor cells undergo brown adipogenesis.

As used herein, “compound-activated” means that the BAT progenitor cell or cells have been treated with the combinations of compounds as described herein. The cells can be autologous, allogeneic, or xenogeneic.

In some embodiments, methods described herein can include implanting a population of compound-activated BAT progenitor cells into a subject. The compound-activated cells can be implanted directly or can be administered in a scaffold, matrix, or other implantable device to which the cells can attach (examples include carriers made of, e.g., collagen, fibronectin, elastin, cellulose acetate, cellulose nitrate, polysaccharide, fibrin, gelatin, self-assembling small peptides, and combinations thereof). In general, the methods include implanting a population of compound-activated BAT progenitor cells comprising a sufficient number of cells to promote increased brown adipocyte mass in the subject, e.g., to increase the amount of brown adipocytes in the subject by at least 1%, e.g., 2%, 5%, 7%, 10%, 15%, 20%, 25% or more. As discussed above, the BAT progenitor cells can be activated by a combination of compounds that comprise (a) bezafibrate or an analog thereof, (b) oxaprozin or an analog thereof, and (c) hFGF7 or analogs thereof, or (d) FGF7 or analogs thereof, for example, human FGF7 and a GLP-1 receptor agonist, or (e) bezafibrate or an analog thereof and oxaprozin or an analog thereof and a GLP-1 receptor agonist.

In general, the subject is a mammal. In some embodiments, the subject is a human subject, e.g., an obese or overweight human subject. In some embodiments, the subject is a non-human mammal, e.g., an experimental animal, a companion animal, or a food animal, e.g., a cow, pig, or sheep that is raised for food. In some embodiments, the methods include evaluating the subject for one or more of: weight, white adipose tissue stores, brown adipose tissue stores, adipose tissue morphology, insulin levels, insulin metabolism, glucose levels, thermogenic capacity, and cold sensitivity. The evaluation can be performed before, during, and/or after the administration of the combination of compounds or compound-activated BAT progenitor cells. For example, the evaluation can be performed at least 1 day, 2 days, 4, 7, 14, 21, 30 or more days before and/or after the administration.

In some embodiments, the methods include one or more additional rounds of treatment with a combination of compounds or implantation of compound-activated BAT progenitor cells, e.g., to increase brown adipocyte mass, e.g., to maintain or further reduce obesity in the subject.

In some embodiments, the disclosure features a composition that includes, either individually or in combination (a) bezafibrate or an analog thereof, (b) oxaprozin or an analog thereof, and (c) hFGF7 or analogs thereof, wherein the hFGF7, bezafibrate and oxaprozin or analogs thereof are present in amounts that, when administered to a patient, are sufficient to treat, prevent, or reduce a metabolic disorder (e.g., obesity or diabetes). Bezafibrate may also be referred to as EGS2026 herein. Oxaprozin may also be referred to as EGS2032 herein.

In other embodiments, the disclosure features a composition that includes, either individually or in combination (a) bezafibrate or an analog thereof, (b) oxaprozin or an analog thereof, (c) hFGF7 or an analog or analogs thereof, and (d) a GLP-1 receptor agonist or agonists, wherein the hFGF7 or analogs thereof, bezafibrate or analogs thereof, oxaprozin or analogs thereof, and a GLP-1 receptor agonist or agonists are present in amounts that, when administered to a patient, are sufficient to treat, prevent, or reduce a metabolic disorder (e.g., obesity or diabetes).

The compositions of the disclosure may be formulated for local administration or systemic administration. If more than one agent is employed, therapeutic agents may be delivered separately or may be admixed into a single formulation. When agents are present in different pharmaceutical compositions, different routes of administration may be employed. Routes of administration for the various embodiments include, but are not limited to, topical, transdermal, and systemic administration (such as, intravenous, intramuscular, subcutaneous, inhalation, rectal, buccal, vaginal, intraperitoneal, intraarticular, ophthalmic or oral administration). As used herein, “systemic administration” refers to all nondermal routes of administration, and specifically excludes topical and transdermal routes of administration. Desirably, the agent of the disclosure and additional therapeutic agents are administered within at least 1, 2, 4, 6, 10, 12, 18, 24 hours, 3 days, 7 days, 10 days, or 14 days apart. The dosage and frequency of administration of each component of the combination can be controlled independently. For example, one compound may be administered three times per day, while the second compound may be administered once per day. Combination therapy may be given in on-and-off cycles that include rest periods so that the patient's body has a chance to recover from any as yet unforeseen side effects. The compounds may also be formulated together such that one administration delivers the compounds (for example, orally as a solid dosage form (e.g., powder, tablet, capsule, liquid capsule, etc.) or as an injectable composition). Optionally, any of the agents of the combination may be administered in a low dosage or in a high dosage, each of which is defined herein.

Generally, when administered to a human, the dosage of bezafibrate and oxaprozin or analogs thereof are provided in amounts that range for a therapeutically effective amount of bezafibrate from about 100 mg to about 400 mg, about 100 mg to about 300 mg, about 200 mg to about 450 mg, or about 5 mg to about 500 mg, and a therapeutically effective amount of oxaprozin that ranges from about 100 mg to about 400 mg, about 100 mg to about 300 mg, about 200 mg to about 450 mg, or about 5 mg to about 500 mg, about 300 mg to about 900 mg, about 300 mg to about 1200 mg, or about 5 mg to about 500 mg for each compound or analogs thereof. Alternatively, bezafibrate or oxaprozin can be dosed in amounts that range from about 0.001 mg to about 2000 mg per day, desirably about 1 mg to about 1000 mg per day, about 200 mg to about 400 mg per day or about 5 mg to about 500 mg per day for each compound or analog thereof. Dosages up to 2000 mg per day for each compound may be necessary. Administration of each drug in the combination can, independently, be one to four times daily for one day to one year, and may even be for the life of the patient. Chronic, long-term administration will be indicated in many cases.

With respect to the GLP-1 receptor agonists, these compounds can be dosed in the following amounts:
Dulaglutide: about 0.5 to about 5 mg per dose (typically once weekly);
Exenatide: about 0.25 to about 15 mcg per dose, typically between 5 and 10 mcg per dose, the amounts being dosed daily;
Exenatide (BYDUREON): about 1 to about 3 mg weekly, preferably about 2 mg weekly;
Liraglutide: about 0.5 to about 2 mg daily, preferably about 1 to about 1.5 mg daily;
Lixisenatide, about 5 to about 30 mcg daily, preferably about 10 mcg to about 20 mcg daily;
Semaglutide (injectable): about 0.25 to about 1.5 mg weekly, preferably about 0.5 to about 1.0 mg weekly;
Semaglutide (oral): about 2.5 to about 20 mg daily, preferably about 7 to about 14 mg daily.
Tirzepatide (injectable): about 1 mg to about 30 mg weekly, preferably about 2.5 mg to about 15 mg weekly;
Danuglipron: about 5 mg to about 200 mg twice a day;

Recommended dosages for GLP-1 receptor agonists are known in the art (see, for example, Hinnen D Glucagon-Like Peptide 1 Receptor Agonists for Type 2 Diabetes. Diabetes Spectr. 2017:30(3):202-210. doi:10.2337/ds16-0026, which is hereby incorporated by reference in its entirety, particularly with reference to Table 1, and https://www.mounjaro.com).

In certain embodiments, FGF7 and other FGF7 analogs can be used. These analogs can be modified FGF7 proteins with modification that include, but are not limited to, PEGylation, fusion to an immunoglobulin (including Fc domains), fusion to Human Serum Albumin (HSA), fusion to human transferrin, genetic fusion of non-exact repeat peptide sequence (XTENylation, also known as rPEG), fusion to CTP peptide from human chorionic gonadotrophin β-subunit (CTP fusion), fusion to elastin-like peptide (ELPylation), fusion with artificial GLK (gelatin-like protein; GLK fusion), fusion to HAP homo-amino acid polymer (HAPylation), and fusion toproline-alanine-serine polymer (PASylation). Non-limiting examples of these analogs include SEQ ID NOs: 1-7 (which are disclosed in U.S. Provisional Application 63/067,675, filed Aug. 19, 2020, having attorney docket number 130204-010400/PRO, and entitled “Analogs of Human Fibroblast Growth Factors”, the disclosure of which is hereby incorporated by reference in its entirety). SEQ ID NO: 8 is the sequence for human FGF7. Analogs of FGF7, including hFGF7, have one or more of the following functional activities: improves parameters of metabolic health, recruitment of brown adipocyte stem/progenitor cells to increase the mass of BAT, increases energy expenditure or metabolic rate with no significant effects on food intake, morphogenesis of epithelium, reepithelialization of wounds, hair development, early lung organogenesis, and other mitogenic activity in keratinocytes.

With respect to FGF7 or analogs thereof, for example human FGF7, the amounts administered to a subject will be on the order of about 0.4 to about 1.5 mg/kg for animals, such as rodents (e.g., mice, etc.). For humans, the dose administered in the context of this disclosure will be about 0.001 to about 0.1 mg/kg, preferably about 0.01 to about 0.08 mg/kg. In some embodiments, these amounts can be reduced by between about 25% and about 80% when used in combination with a GLP-1 receptor agonist. In certain embodiments, analogs of FGF7 can be administered at higher doses than human FGF7. For example, the amounts of FGF7 analogs administered to a subject will be on the order of about 0.4 to about 10 mg/kg or about 3 to about 5 mg/kg for animals, such as rodents (e.g., mice, etc.). For humans, the dose administered in the context of this disclosure will be about 0.001 to about 1 mg/kg, preferably about 0.01 to about 0.5 mg/kg.

In some embodiments, the GLP-1 receptor agonists disclosed herein, when used in combination with either FGF7 or analogs thereof or the combination of bezafibrate or an analog thereof and oxaprozin or an analog thereof, can be administered in reduced amounts, for example amounts that are between about 25% and about 80% lower than a standard dose.

The therapeutic agents of the disclosure may be admixed with additional active or inert ingredients, e.g., in conventional pharmaceutically acceptable carriers. A pharmaceutical carrier can be any compatible, non-toxic substance suitable for the administration of the compositions of the present disclosure to a mammal. Pharmaceutically acceptable carriers include, for example, water, saline, buffers, and other compounds described for example in the Merck Index, Merck & Co., Rahway, N.J. Slow-release formulation or a slow release apparatus may be also be used for continuous administration.

If more than one agent is employed, each agent may be formulated in a variety of ways that are known in the art. Desirably, the agents are formulated together for the simultaneous or near simultaneous administration of the agents. Such co-formulated compositions can include two or three agents formulated together in the same pill, tablet, capsule, liquid, etc. It is to be understood that, when referring to the formulation of such combinations, the formulation technology employed is also useful for the formulation of the individual agents of the combination, as well as other combinations of the disclosure. By using different formulation strategies for different agents, the pharmacokinetic profiles for each agent can be suitably matched.

The methods of this disclosure may also be used prophylactically, in patients who are an increased risk of developing obesity, diabetes or a condition associated with obesity or diabetes such as insulin resistance. Risk factors include for example, family history of diabetes or obesity or associated conditions, quality of nutrition, level of physical activity, presence of molecular markers of obesity or diabetes, history of bariatric surgery for obesity with or without co-morbidities, age, race, or sex. Patients affected with other non-related disorders may also be predisposed to secondary obesity or diabetes. In certain embodiments, the compositions and methods of this disclosure may be used in patients to maintain a weight, particularly in patients that were formerly obese and/or that have undergone bariatric surgery.

The disclosure also features a method for treating, preventing, or reducing a metabolic disorder in a patient in need thereof by administering to the patient (i) bezafibrate or an analog thereof and (ii) oxaprozin or an analog thereof and (iii) a GLP-1 receptor agonist, wherein the bezafibrate and oxaprozin or analogs thereof and GLP-1 receptor agonist are administered in amounts that together are sufficient to treat, prevent, or reduce a metabolic disorder.

The disclosure also features a method for treating, preventing, or reducing a metabolic disorder in a patient in need thereof by administering to the patient (i) bezafibrate or an analog thereof and (ii) ozagrel or an analog thereof and (iii) a GLP-1 receptor agonist, wherein the bezafibrate and ozagrel or analogs thereof and GLP-1 receptor agonist are administered in amounts that together are sufficient to treat, prevent, or reduce a metabolic disorder.

The disclosure also features a method for treating, preventing, or reducing a metabolic disorder in a patient in need thereof by administering to the patient (i) bezafibrate or an analog thereof and (ii) zaltoprofen or an analog thereof and (iii) a GLP-1 receptor agonist, wherein the bezafibrate and zaltoprofen or analogs thereof and GLP-1 receptor agonist are administered in amounts that together are sufficient to treat, prevent, or reduce a metabolic disorder.

The individually or separately formulated agents can be packaged together as a kit. Non-limiting examples include kits that contain, e.g., two pills and an injectable solution, two pills and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blisterpacks, bottles, vials, syringes, tubes, and the like.

In some aspects the treated metabolic disease may be obesity, overweight, type II diabetes, insulin resistance, hyperinsulinemia, hyperglycemia, pre-diabetes, hypertension, hyperlipidemia, hepatosteatosis, fatty liver, non-alcoholic fatty liver disease, hyperuricemia, polycystic ovarian syndrome, acanthosis nigricans, hyperphagia, endocrine abnormalities, triglyceride storage disease, Bardet-Biedl syndrome, Laurence-Moon syndrome, Prader-Willi syndrome, neurodegenerative diseases, and Alzheimer's disease.

In other embodiments, compositions may be used to activate isolated BAT progenitor cells that are then used for treatment of a subject, including a human subject.

In one example, we propose that the administration of FGF7 or analogs thereof, bezafibrate and oxaprozin to a patient having a metabolic disorder such as obesity or diabetes within 14 days of each other will treat, prevent, or reduce the metabolic disorder.

The agents are desirably administered within 10 days of each other, more desirably within seven days of each other, and even more desirably within twenty-four hours of each other, one hour of each other, or even simultaneously (i.e., concomitantly). If desired, any or all of the agents may be administered in low dosage (for example, in an amount that is about 10 to about 75% lower than the dose of the agent approved for use in a subject, for example humans).

By “treating” is meant ameliorating a condition. The terms “treatment, treating, treat” or equivalents of these terms refer to healing, alleviating, relieving, altering, remedying, ameliorating, improving, or affecting the condition or the symptoms of a subject as compared with an equivalent untreated control, such reduction or degree of amelioration is at least 5%, 10%, 20%, 40%, 50%, 60%, 80%, 90%, 95%, or 100% as measured by any standard technique.

In the context of compositions containing amounts of ingredients where the terms “about” is used, these compositions contain the stated amount of the ingredient with a variation (error range) of 0-10% around the value (X±10%). In other contexts the term “about” is provides a variation (error range) of 0-10% around a given value (X±10%).

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

A patient who is being treated for a metabolic disorder is one who a medical practitioner has diagnosed as having such a condition. Diagnosis may be performed by any suitable means, such as those described herein. A patient in whom the development of diabetes or obesity is being prevented may or may not have received such a diagnosis. One in the art will understand that patients of the disclosure may have been subjected to standard tests or may have been identified, without examination, as one at high risk due to the presence of one or more risk factors, such as family history, obesity, particular ethnicity (e.g., African Americans and Hispanic Americans), gestational diabetes or delivering a baby that weighs more than nine pounds, hypertension, having a pathological condition predisposing to obesity or diabetes, high blood levels of triglycerides, high blood levels of cholesterol, presence of molecular markers (e.g., presence of autoantibodies), a history of bariatric surgery, and age (over 45 years of age). An individual is considered obese when their weight is 20% (25% in women) or more over the maximum weight desirable for their height. An adult who is more than 100 pounds overweight is considered to be morbidly obese. Obesity is also defined as a body mass index (BMI) over 30 kg/m2 and morbid obesity as a body mass index (BMI) over 40 kg/m2.

By “a metabolic disorder” is meant any pathological condition resulting from an alteration in a patient's metabolism. Such disorders include those resulting from an alteration in glucose homeostasis resulting, for example, in hyperglycemia. According to this disclosure, an alteration in glucose levels is typically an increase in glucose levels by at least 5%, 10%, 20%, 30%, 40%, 50%, 60%, 70%, 80%, 90%, 100%, 200%, 300%, or even 400% relative to such levels in a healthy individual. Metabolic disorders include obesity and diabetes (e.g., diabetes type I, diabetes type II, MODY, and gestational diabetes), dyslipidemia, hepatosteatosis, and endocrine deficiencies of aging.

By “reducing glucose levels” is meant reducing the level of glucose by at least 10%, 20%, 30%, 40%, 50%, 60%, 70%, 80%, 90%, 95%, or 100% relative to an untreated control. Desirably, glucose levels are reduced to normoglycemic levels, i.e., between 150 to 60 mg/dL, between 140 to 70 mg/dL, between 130 to 70 mg/dL, between 125 to 80 mg/dL, and preferably between 120 to 80 mg/dL.

By “patient” or “subject” is meant any animal (e.g., a human), including horses, dogs, cats, pigs, goats, rabbits, hamsters, monkeys, guinea pigs, rats, mice, lizards, Snakes, sheep, cattle, fish, and birds.

By “an amount sufficient” is meant the amount of a compound, alone or in combination with another therapeutic regimen, required to treat, or reduce, or prevent a metabolic disorder such as diabetes or obesity in a clinically relevant manner. A sufficient amount of an active compound used to practice the present disclosure for therapeutic treatment of metabolic disorders varies depending upon the manner of administration, the age, body weight, and general health of the mammal or patient. Ultimately, the prescribers will decide the appropriate amount and dosage regimen. Additionally, an effective amount may be an amount of compound in the combination of the disclosure that is safe and efficacious in the treatment of a patient having a metabolic disorder such as diabetes over each agent alone as determined and approved by a regulatory authority (such as the U.S. Food and Drug Administration).

By “more effective” is meant that a treatment exhibits greater efficacy, or is less toxic, safer, more convenient, or less expensive than another treatment with which it is being compared. Efficacy may be measured by a skilled practitioner using any standard method that is appropriate for a given indication.

Compounds useful in the disclosure include those described herein in any of their pharmaceutically acceptable forms, including isomers such as diastereomers and enantiomers, salts, esters, solvates, and polymorphs thereof, as well as racemic mixtures and pure isomers of the compounds described herein.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, database entries, and other references mentioned herein are incorporated by reference in their entirety. In case of conflict, the present specification, including definitions, will control.

Bezafibrate (2-(4-{2-[(4-chlorobenzoyl)amino]ethyl}phenoxy)-2-methylpropanoic acid) has the following structure:

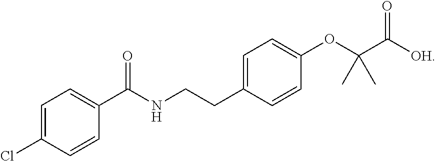 [SMILES: CC(C)(Oc1ccc(CCNC(=O)c2ccc(Cl)cc2)cc1)C(=O)O]

Oxaprozin (3-(4,5-Diphenyloxazol-2-yl)propionic acid) has the following structure:

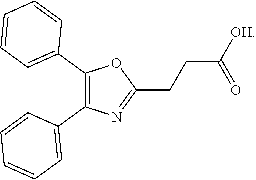 [SMILES: O=C(O)CCc1nc(-c2ccccc2)c(-c2ccccc2)o1]

### EXAMPLES

Aspects of the present teachings may be further understood in light of the following examples, which should not be construed as limiting the scope of the present teachings in any way.

### Example 1: The Combination of Bezafibrate and Oxaprozin Plus Semaglutide Induces Greater than Expected Body Weight Loss, Body Fat Loss, and Reduction in Hepatosteatosis in a Mouse Model of Obesity and Pre-Diabetes

Applicants previously demonstrated that agents which enhance energy expenditure, such as those promoting the differentiation of human or non-human brown adipocyte progenitor cells into brown adipocytes, i.e., an agent that recruits brown adipocytes or BAT in vivo, can cause improvement in parameters of metabolic health in obese individuals or animals or diabetic individuals or animals or individuals of animals with other metabolic conditions, such as decreases in body weight, body fat content, plasma levels of leptin, glucose, insulin, and an index of insulin resistance, the Homeostatic Model Assessment for Insulin Resistance (HOMA-IR). The HOMA-IR equals the plasma insulin [microIU/ml]×plasma glucose [mM])/22.5).

These findings were made in the commonly used mouse model of environmental obesity and pre-diabetes, or insulin resistance, the Diet-Induced Obese (DIO) mouse. In order to uncover possible greater effects between agents that increase energy expenditure, for example by recruiting brown adipocytes, and an agent that decreases body weight by reducing food intake, we investigated the effects of the combination of bezafibrate and oxaprozin and the GLP-1 receptor agonist semaglutide.

Bezafibrate and oxaprozin both recruit brown adipocytes and are known, or are believed, to affect different molecular targets and intracellular signaling pathways. Semaglutide decreases food intake though activation of the GLP-1 receptor.

### Materials and Methods

### Animal Studies

Obesity and insulin resistance, an early stage in the development of type 2 diabetes (also known as pre-diabetes), was induced in C57Bl/6 mice by feeding the mice with a high fat diet (Research Diets, Cat #D12492, 60% fat kcal) for 12 weeks starting at 6 weeks of age, and throughout the period of compound dosing. The mice were housed at 22-23° C. and abundant nestlets/bedding material was provided to allow the animals to maintain a microenvironment close to their thermoneutrality of 28-30° C., starting 2 weeks prior to the dosing period and for the full dosing period with a 12 h/12 h light/dark cycle. These environmental conditions are understood by those skilled in the art to reduce the stimulus for maintaining BAT, a thermogenic tissue that is recruited physiologically by cold stimulus, and permit a wider window for observing effects related to BAT recruitment.

Mice were dosed once per day by oral gavage (100 μl per mouse) with vehicle (PBS+0.5% CMC+0.1% Tween-80) alone or with bezafibrate (60 mg/kg)+oxaprozin (50 mg/kg) (also referred to as EGS2632) dissolved in the vehicle, for 34 days. In addition, the mice received every 3 days by intraperitoneal injection (100 μl per mouse) either vehicle (9.6 mg/ml mannitol, 4.8 mg/ml sucrose, 0.37 mg/ml L-histidine, 0.025 mg/ml polysorbate 20 (Tween 20, CAS #9005-64-5), pH adjusted to 7.4 with HCl) or semaglutide (0.012 mg/kg=3 nmol/kg) dissolved in the vehicle.

Body weight was recorded every day, and body composition (fat and lean mass with EchoMRI) was assessed at the end of the study (University of Cincinnati Mouse Metabolic Phenotyping Center). At the end of the dosing period, animals were euthanized by CO₂, and a piece (approximately 50 mg) of liver was collected and frozen for triglyceride quantification (Triglyceride Quantification Kit, Sigma-Aldrich, St. Louis, MO). Blood plasma was isolated from submandibular blood collected from animals fasted for 6 hours at baseline and at the end of the dosing period. Plasma glucose, insulin and leptin levels were assessed at baseline and at the end of the dosing period (University of Cincinnati Mouse Metabolic Phenotyping Center). Insulin sensitivity was determined using the homeostasis model assessment of insulin resistance (HOMA-IR). HOMA-IR=(plasma insulin [microIU/ml]×plasma glucose [mM])/22.5.

### Statistical Analysis

Data from in vivo mouse studies are presented as means±SEM. Significance values were evaluated based on the Z-test with normal approximations. For body fat and liver fat, since the distributions of these values were both skewed, we used the log-transformed values to better approximate the normal distribution. To assess synergistic effects of A and B, the combination A+B was compared to the sum of A alone and B alone. To quantify synergistic effects, we used percent changes from baseline when baseline data were available and used the difference from vehicle for parameters without baseline data.

### Results

Applicants tested the effects of bezafibrate (60 mg/kg)+oxaprozin (50 mg/kg), semaglutide (0.012 mg/kg) alone, and the combination of bezafibrate (60 mg/kg)+oxaprozin (50 mg/kg) with semaglutide (0.012 mg/kg) in DIO mice over 34 days of dosing on parameters of metabolic health.

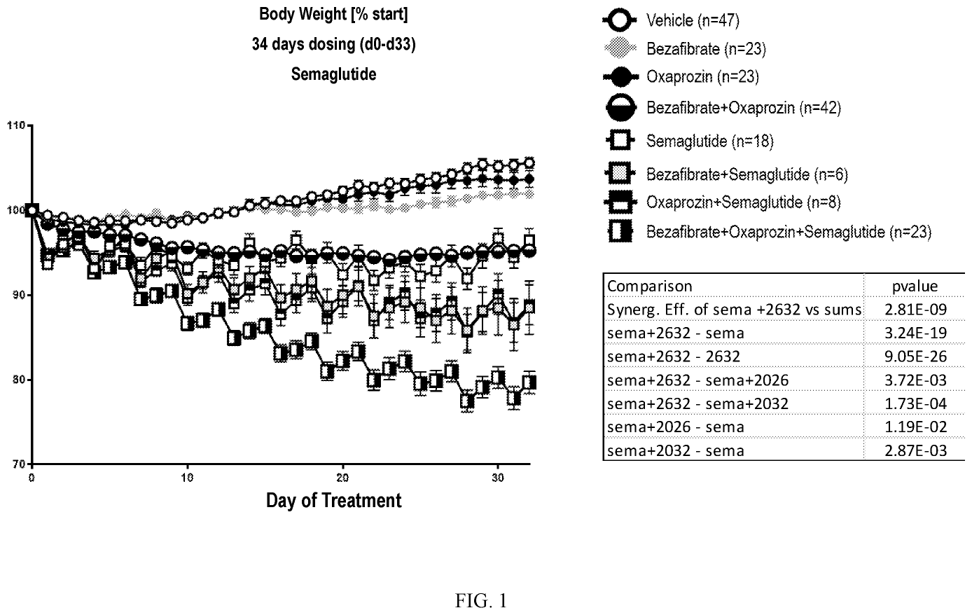

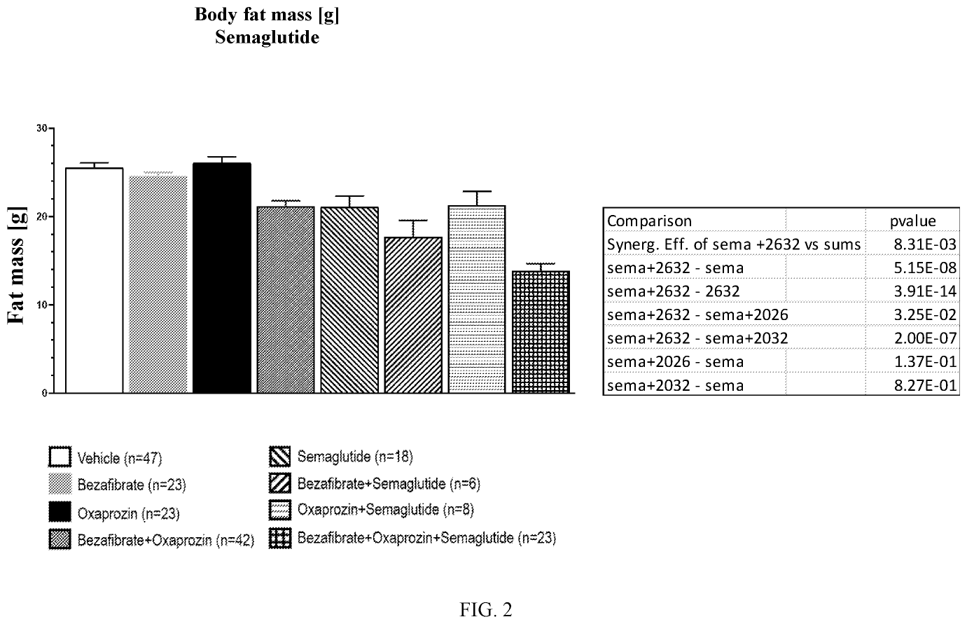

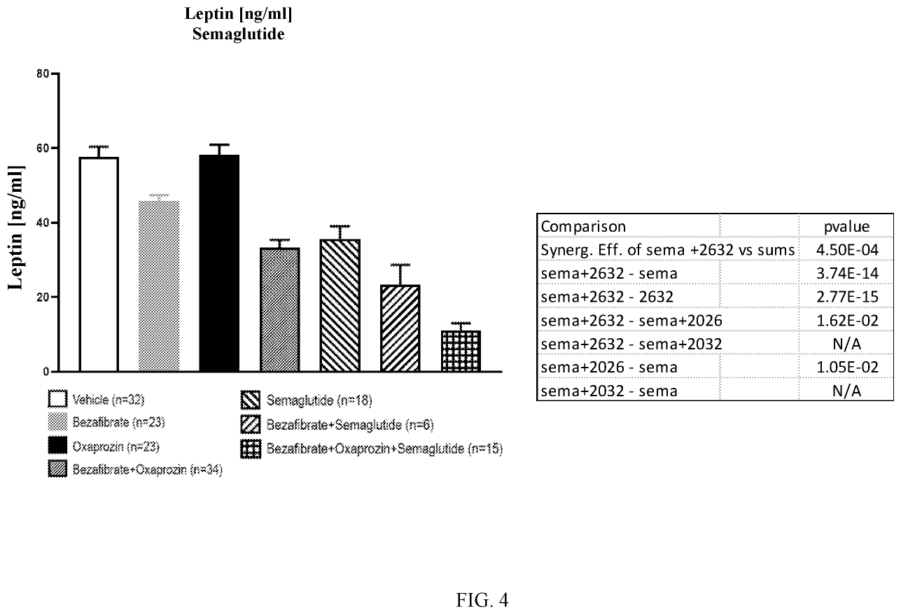

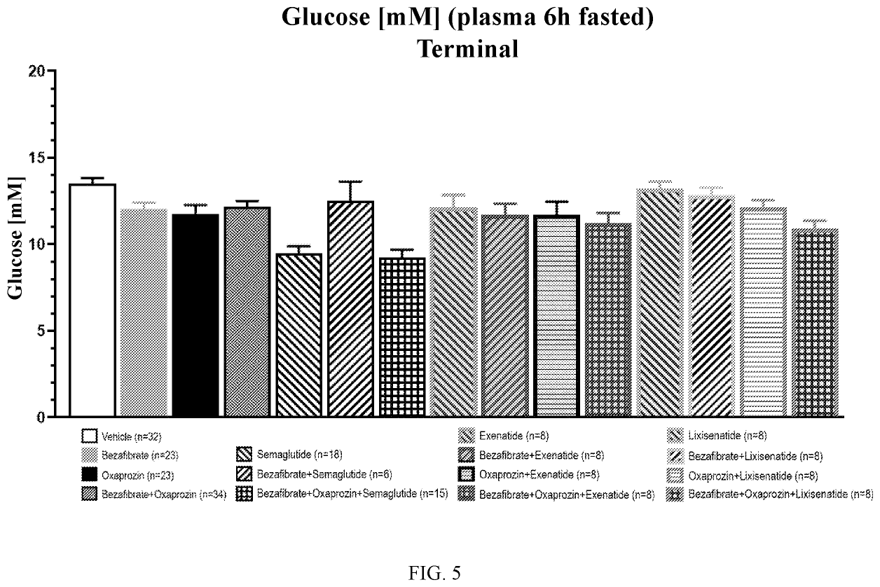

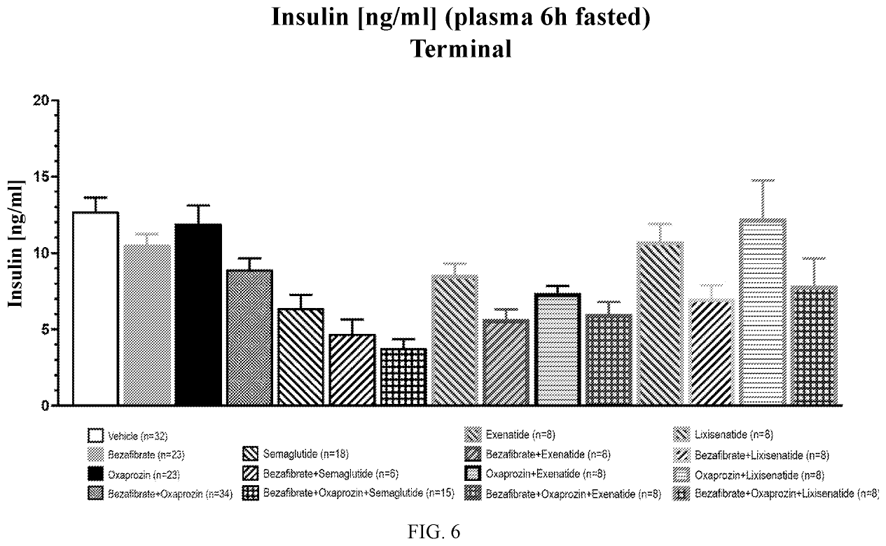

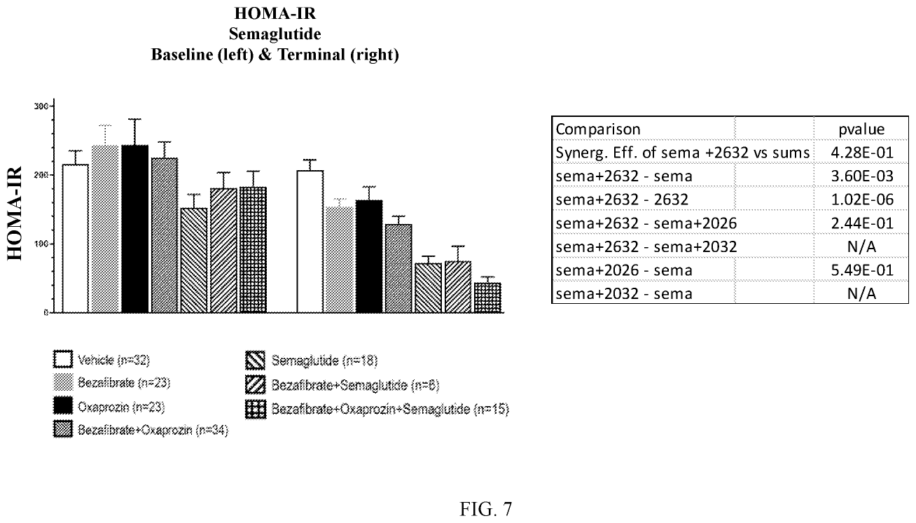

Bezafibrate (60 mg/kg)+oxaprozin (50 mg/kg) over 34 days, compared to treatment with vehicle, induced significant decreases in body weight of 11.2% (FIG. 1, pvalue 9.9e-32), body fat mass (FIG. 2, pvalue 1.5e-07), plasma leptin (FIG. 4, pvalue 3.5e-14), plasma insulin (FIG. 6, 4.24e-05) and insulin resistance index (HOMA-IR) (FIG. 7, pvalue 1.2e-05).

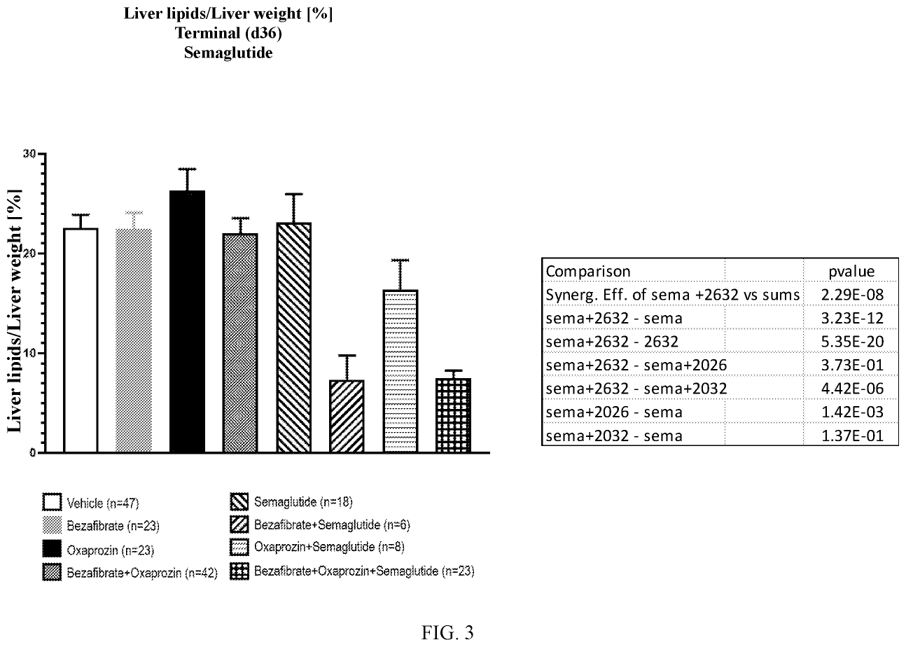

In addition, the combination of bezafibrate (60 mg/kg)+oxaprozin (50 mg/kg) with semaglutide (0.012 mg/kg) caused further decreases beyond semaglutide alone in several of these metabolic parameters. Surprisingly, the weight loss-inducing effect of the combination with semaglutide (FIG. 1) as well as the effects on body fat (FIG. 2) and hepatic fat (FIG. 3) were greater than additive. In fact, bezafibrate+oxaprozin+semaglutide produced highly significant reduction in body weight in DIO mice over 34 days (p<0.0001). For body weight, we evaluated drug effects based on the percent change from baseline on day 35 (generally), however in the case of semaglutide, which was dosed every 3 days, we used the average weights on days 33-35. The observed synergistic effect was 12.0 percent greater reduction in body weight beyond the sum of the EGS2632 and semaglutide groups as a result of concurrent treatment with EGS2632 with semaglutide (pvalue 2.81e-09).

Comparing the combination of bezafibrate (60 mg/kg)+oxaprozin (50 mg/kg) with semaglutide to the sum of the bezafibrate/ozaprozin and semaglutide alone groups also showed an additional reduction in liver fat as a result of treatment with bezafibrate/ozaprozin with semaglutide (pvalue 2.3e-08); there is furthermore an additional reduction in body fat as a result of treatment with EGS2632 with semaglutide (pvalue 8.3e-03).

Bezafibrate+oxaprozin with semaglutide also synergistically lowered leptin levels, with a 28.5 percent reduction from baseline beyond the sum of the bezafibrate+oxaprozin and semaglutide groups (pvalue 4.5e-04). Bezafibrate+oxaprozin with semaglutide reduced HOMA-IR beyond the reduction achieved with semaglutide alone (pvalue 3.6e-03).

In summary, the magnitude of the effects of the combination of bezafibrate+oxaprozin and semaglutide on body weight and other metabolic parameters could not have been anticipated based on the known effects of these individual agents. In fact, surprisingly, synergistic effects were observed on several parameters of metabolic status.

### Example 2: The Combination of Human FGF7 and Semaglutide Induces Body Weight Loss, an Increase in Insulin Sensitivity, and Improvement in Blood Glucose Homeostasis in a Mouse Model of Obesity and Diabetes

As above, applicants previously demonstrated that agents which enhance energy expenditure, such as hFGF7, which promotes the differentiation of human or non-human brown adipocyte progenitor cells into brown adipocytes, i.e., recruits brown adipocytes or BAT in vivo, can cause improvement in parameters of metabolic health in obese individuals or animals or diabetic individuals or animals or individuals of animals with other metabolic conditions, such as decreases in body weight, body fat content, plasma levels of leptin, glucose, insulin, and an index of insulin resistance, the Homeostatic Model Assessment for Insulin Resistance (HOMA-IR). The HOMA-IR equals the plasma insulin [microIU/ml]×plasma glucose [mM])/22.5).

These findings were made in the commonly used mouse model of environmental obesity and pre-diabetes, or insulin resistance, the Diet-Induced Obese (DIO) mouse. In order to uncover possible greater effects between agents that increase energy expenditure, for example by recruiting brown adipocytes, and an agent that decreases body weight by reducing food intake, we investigated the effects of the combination of hFGF7 and the GLP-1 receptor agonist semaglutide.

hFGF7 recruits brown adipocytes. Semaglutide decreases food intake though activation of the GLP-1 receptor.

### Materials and Methods

### Animal Studies

Obesity and insulin resistance, an early stage in the development of type 2 diabetes (also known as pre-diabetes), was induced in C57Bl/6 mice by feeding the mice with a high fat diet (Research Diets, Cat #D12492, 60% fat kcal) for 12 weeks starting at 6 weeks of age, and throughout the period of compound dosing. The mice were housed at 22-23° C. and abundant nestlets/bedding material was provided to allow the animals to maintain a microenvironment close to their thermoneutrality of 28-30° C., starting 2 weeks prior to the dosing period and for the full dosing period with a 12 h/12 h light/dark cycle. These environmental conditions are understood by those skilled in the art to reduce the stimulus for maintaining BAT, a thermogenic tissue that is recruited physiologically by cold stimulus, and permit a wider window for observing effects related to BAT recruitment.

Mice were dosed once per day by intraperitoneal injection (100 μl per mouse) with vehicle (9.6 mg/ml mannitol, 4.8 mg/ml sucrose, 0.37 mg/ml L-histidine, 0.025 mg/ml polysorbate 20 (Tween 20, CAS #9005-64-5), pH adjusted to 7.4 with HCl) alone or recombinant hFGF7 (1 mg/kg, also referred to as EGS0501 in corresponding figures) dissolved in the vehicle for 28 days. In addition, some mice also received semaglutide (0.012 mg/kg=3 nmol/kg) every 3 days by intraperitoneal injection (100 μl per mouse) dissolved in the vehicle.

Body weight was recorded every day and body composition (fat and lean mass with EchoMRI) was assessed at the end of the study (University of Cincinnati Mouse Metabolic Phenotyping Center). At the end of the dosing period, animals were fasted for 6 hours and euthanized by CO₂, blood was collected, and plasma was isolated and frozen at −20° C. Plasma glucose, insulin and leptin levels were assessed at baseline and at the end of the dosing period (mice were fasted for 6 hours before all plasma collection) (University of Cincinnati Mouse Metabolic Phenotyping Center). Insulin sensitivity was determined by HOMA-IR.

### Statistical Analysis

Data from in vivo mouse studies are presented as means±SEM. Significance values were evaluated based on the unpaired two-tailed t-test versus vehicle using GraphPad Prism version 7 or 8 (GraphPad Software, San Diego, CA). To assess synergistic effects of A and B, the combination A+B was compared to the sum of A alone and B alone. To quantify synergistic effects, we used percent changes from baseline when baseline data were available and used the difference from vehicle for parameters without baseline data.

### Results

Applicants tested the effects of hFGF7 alone at 1 mg/kg and at 0.5 mg/kg, semaglutide alone, and each dose of hFGF7 combined with semaglutide (0.012 mg/kg), in DIO mice over 28 days of dosing on parameters of metabolic health.

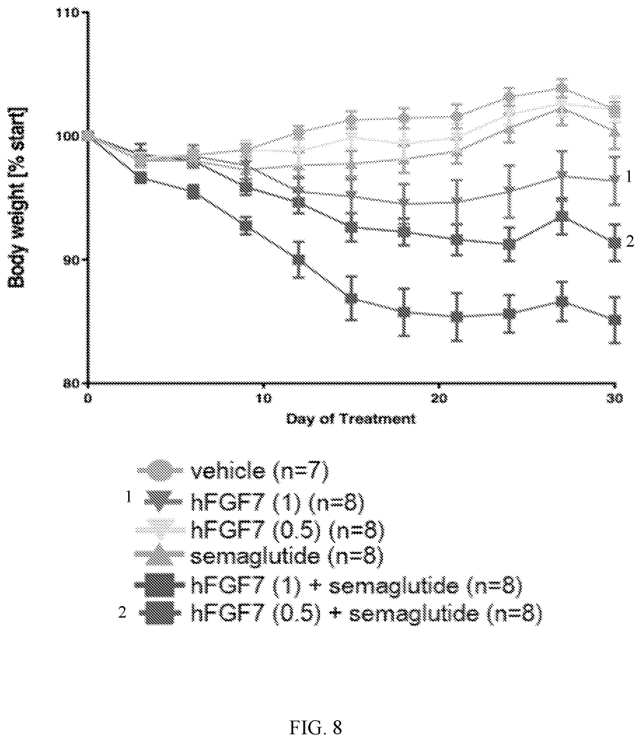

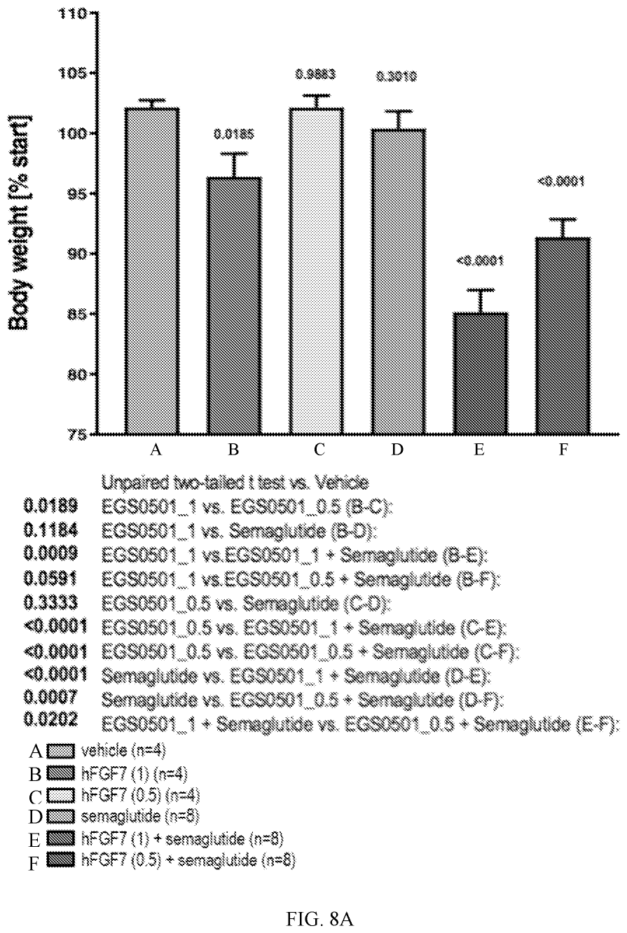

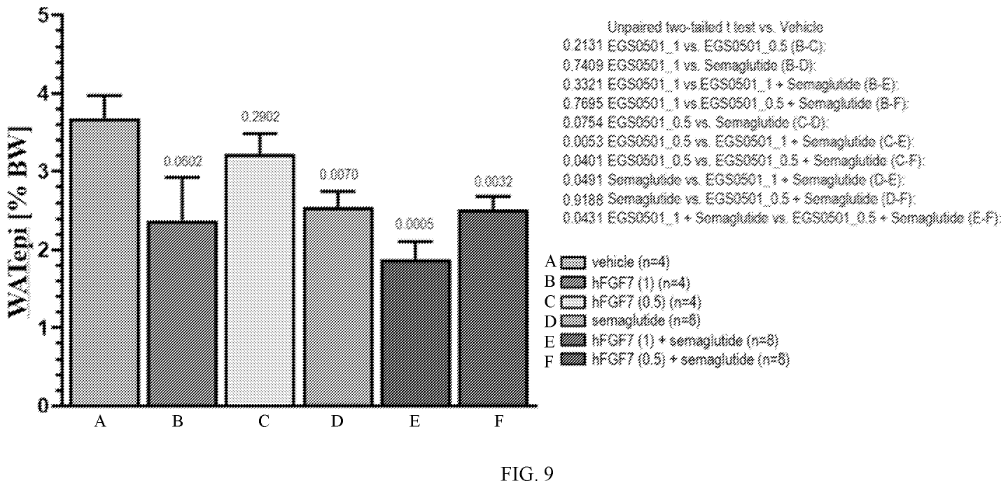

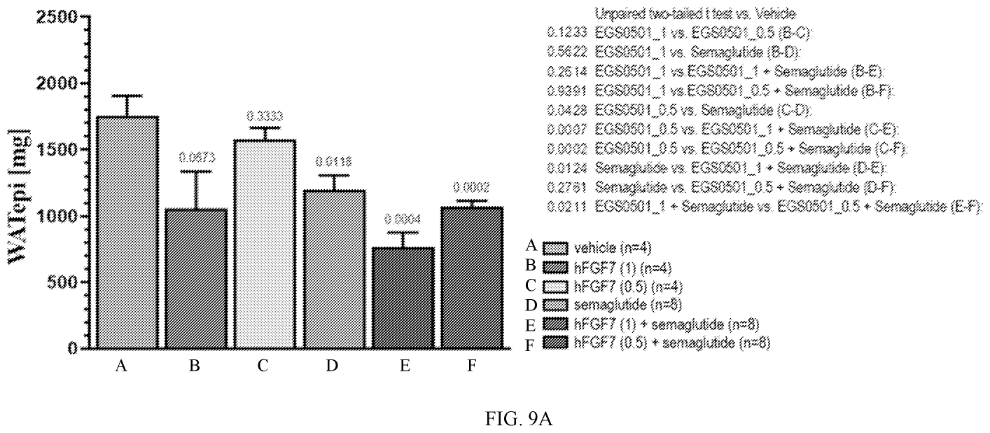

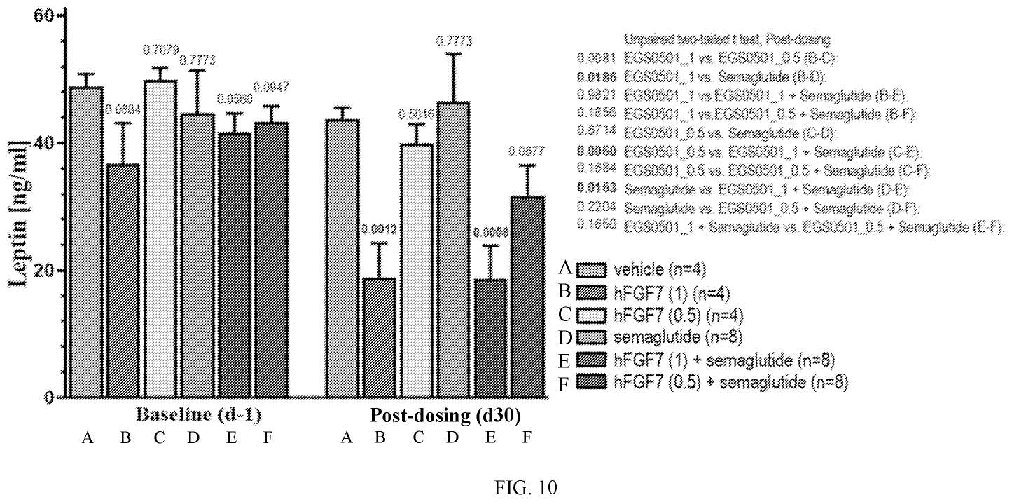

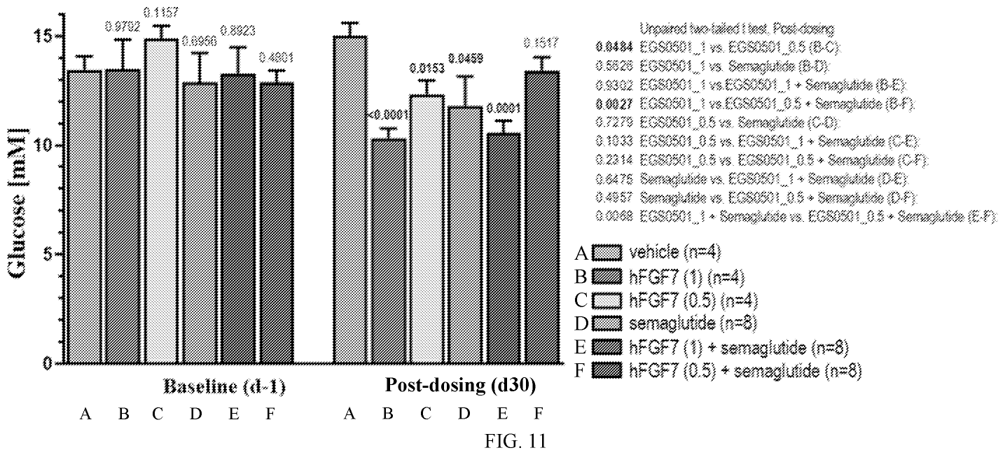

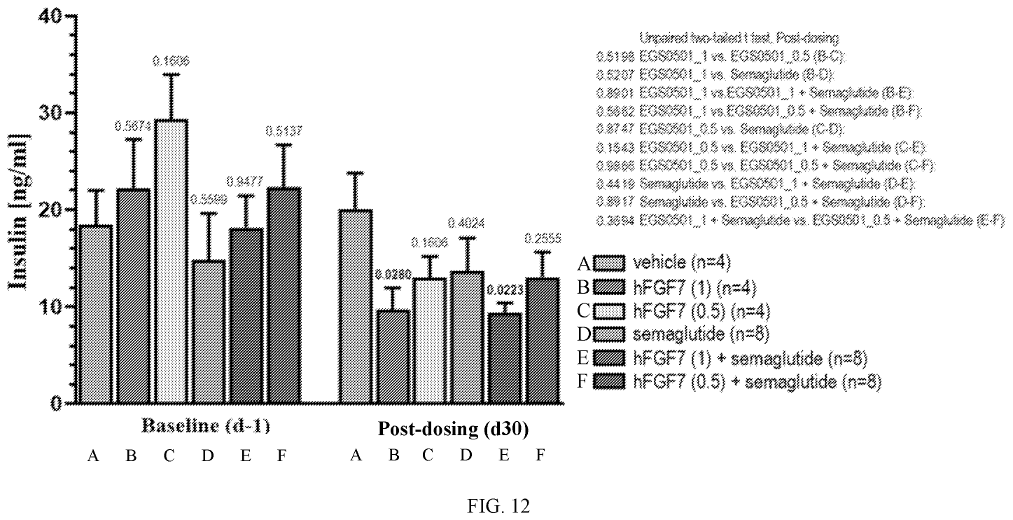

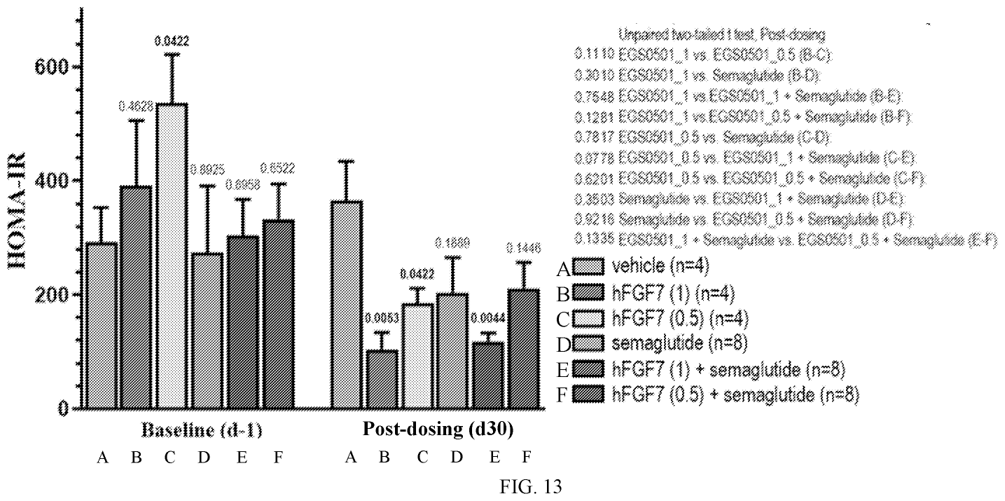

Treatment of DIO mice with hFGF7 at 1 mg/kg over 28 days, compared to treatment with vehicle, induced significant decreases in body weight (FIGS. 8 and 8A), epididymal white adipose (WATepi) depot weight (an index of body fat mass) (FIGS. 9 and 9A), plasma leptin (FIG. 10), plasma glucose (FIG. 11), plasma insulin (FIG. 12), and insulin resistance index (HOMA-IR) (FIG. 13). The lower dose of hFGF7 (0.5 mg/kg) had a significant effect only on plasma glucose (FIG. 11) and insulin resistance index (HOMA-IR) (FIG. 13).

Semaglutide alone had a significant effect only on epididymal white adipose (WATepi) depot weight (FIGS. 9 and 9A) and plasma glucose (FIG. 11).

Applicants found that the combination of hFGF7 at 1 mg/kg with semaglutide further reduced body weight (FIGS. 8 and 8A). Surprisingly, the weight loss-inducing effect of the combination was synergistic (i.e., greater than additive). Moreover, the combination of hFGF7 at 0.5 mg/kg with semaglutide also caused greater reductions in body weight than the sum of the 2 agents (FIGS. 8 and 8A), i.e., the weight loss-inducing effect of the combination again was synergistic.

We found that hFGF7 (1 mg/kg)+semaglutide produced highly significant reduction in body weight in DIO mice over 28 days (p<0.0001). To assess synergistic effects, the percent change from baseline with the combination was compared with the sum of the percent changes from baseline of the hFGF7 and semaglutide groups using two-sided Z-tests. The observed synergistic effect was 5.8% beyond the sum of the two individual drugs (p=0.022) when hFGF7 was used at 0.5 mg/kg/day and 6% (p=0.037) when hFGF7 was used at 1 mg/kg/day.

### Example 3: The Combination of Bezafibrate and Oxaprozin Plus Exenatide Induces Greater than Expected Body Weight Loss, Body Fat Loss, and Reduction in Hepatosteatosis in a Mouse Model of Obesity and Pre-Diabetes

Applicants previously demonstrated that agents which enhance energy expenditure, such as those promoting the differentiation of human or non-human brown adipocyte progenitor cells into brown adipocytes, i.e., an agent that recruits brown adipocytes or BAT in vivo, can cause improvement in parameters of metabolic health in obese individuals or animals or diabetic individuals or animals or individuals of animals with other metabolic conditions, such as decreases in body weight, body fat content, hepatosteatosis, plasma levels of leptin, glucose, insulin, and an index of insulin resistance, the Homeostatic Model Assessment for Insulin Resistance (HOMA-IR). The HOMA-IR equals the plasma insulin [microIU/ml]×plasma glucose [mM])/22.5).

These findings were made in the commonly used mouse model of environmental obesity and pre-diabetes, or insulin resistance, the Diet-Induced Obese (DIO) mouse. In order to uncover possible greater effects between agents that increase energy expenditure, for example by recruiting brown adipocytes, and an agent that decreases body weight by reducing food intake, we investigated the effects of the combination of bezafibrate and oxaprozin and the GLP-1 receptor agonist exenatide. This is the second of 4 GLP-1 receptor agonists we investigated in combination with bezafibrate and oxaprozin.

Bezafibrate and oxaprozin both recruit brown adipocytes and are known, or are believed, to affect different molecular targets and intracellular signaling pathways. Exenatide decreases food intake though activation of the GLP-1 receptor.

### Materials and Methods

### Animal Studies

Obesity and insulin resistance, an early stage in the development of type 2 diabetes (also known as pre-diabetes), was induced in C57Bl/6 mice by feeding the mice with a high fat diet (Research Diets, Cat #D12492, 60% fat kcal) for 12 weeks starting at 6 weeks of age, and throughout the period of compound dosing. The mice were housed at 22-23° C. and abundant nestlets/bedding material was provided to allow the animals to maintain a microenvironment close to their thermoneutrality of 28-30° C., starting 2 weeks prior to the dosing period and for the full dosing period with a 12 h/12 h light/dark cycle. These environmental conditions are understood by those skilled in the art to reduce the stimulus for maintaining BAT, a thermogenic tissue that is recruited physiologically by cold stimulus, and permit a wider window for observing effects related to BAT recruitment.

Mice were dosed once per day by oral gavage (100 μl per mouse) with vehicle (PBS+0.5% CMC+0.1% Tween-80) alone or with bezafibrate (60 mg/kg)+oxaprozin (50 mg/kg) (also referred to as EGS2632) dissolved in the vehicle, for 34 days. In addition, the mice received every day by intraperitoneal injection (100 μl per mouse) either vehicle (9.6 mg/ml mannitol, 4.8 mg/ml sucrose, 0.37 mg/ml L-histidine, 0.025 mg/ml polysorbate 20 (Tween 20, CAS #9005-64-5), pH adjusted to 7.4 with HCl) or exenatide ((0.05 mg/kg) dissolved in the vehicle.

Body weight was recorded every day, body composition (fat and lean mass with EchoMRI) was assessed at the end of the study (University of Cincinnati Mouse Metabolic Phenotyping Center). At the end of the dosing period, animals were euthanized by CO₂, and a piece (approximately 50 mg) of liver was collected and frozen for triglyceride quantification (Triglyceride Quantification Kit, Sigma-Aldrich, St. Louis, MO). Blood plasma was isolated from submandibular blood collected from animals fasted for 6 hours at baseline and at the end of the dosing period. Plasma glucose, insulin and leptin levels were assessed at baseline and at the end of the dosing period (University of Cincinnati Mouse Metabolic Phenotyping Center). Insulin sensitivity was determined using the homeostasis model assessment of insulin resistance (HOMA-IR).

### Statistical Analysis

Data from in vivo mouse studies are presented as means±SEM. Significance values were evaluated based on the Z-test with normal approximations. For body fat and liver fat, since the distributions of these values were both skewed, we used the log-transformed values to better approximate the normal distribution. To assess synergistic effects of A and B, the combination A+B was compared to the sum of A alone and B alone. To quantify synergistic effects, we used percent changes from baseline when baseline data were available and used the difference from vehicle for parameters without baseline data.

### Results

Applicants tested the effects of bezafibrate (60 mg/kg)+oxaprozin (50 mg/kg), exenatide (0.05 mg/kg) alone, and the combination of bezafibrate (60 mg/kg)+oxaprozin (50 mg/kg) with exenatide (0.05 mg/kg) in DIO mice over 34 days of dosing on parameters of metabolic health.

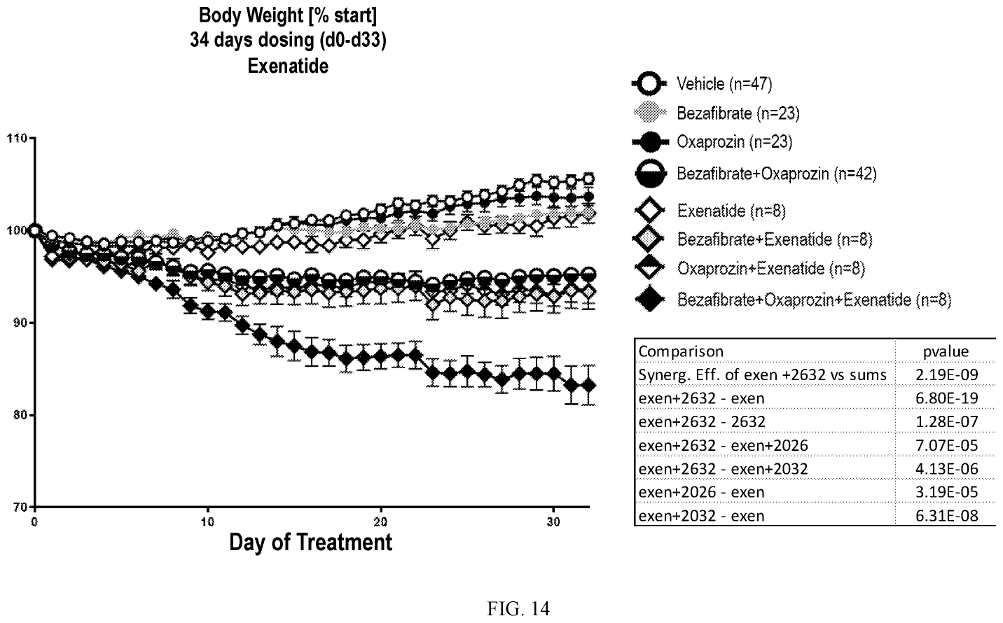

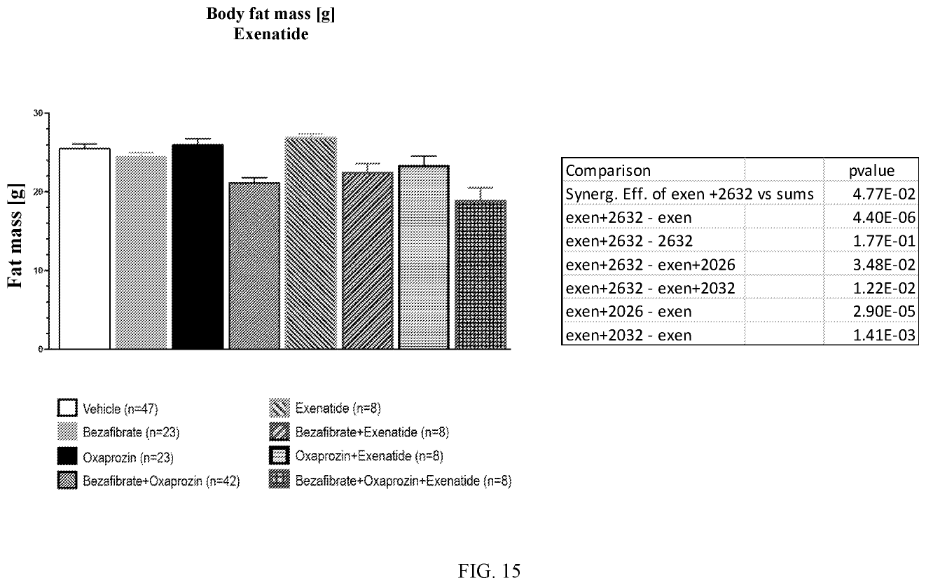

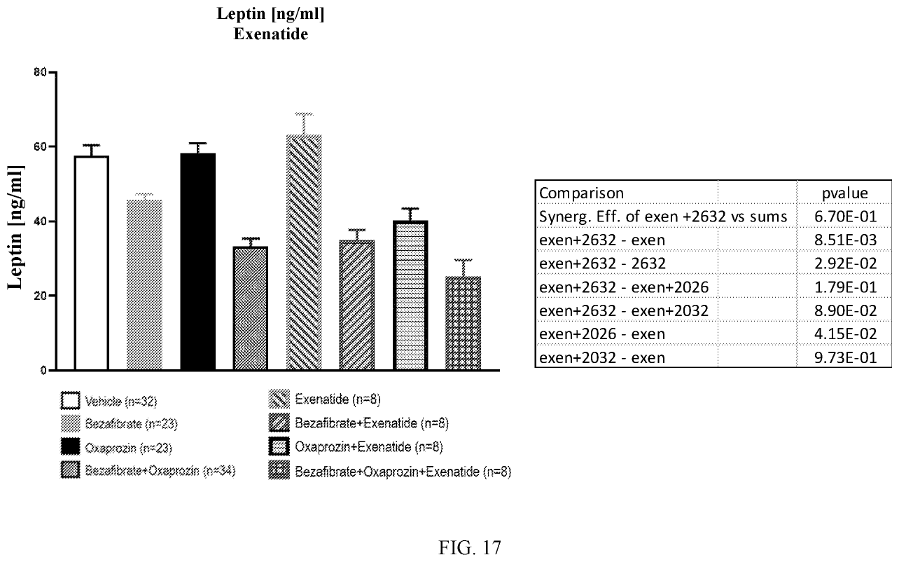

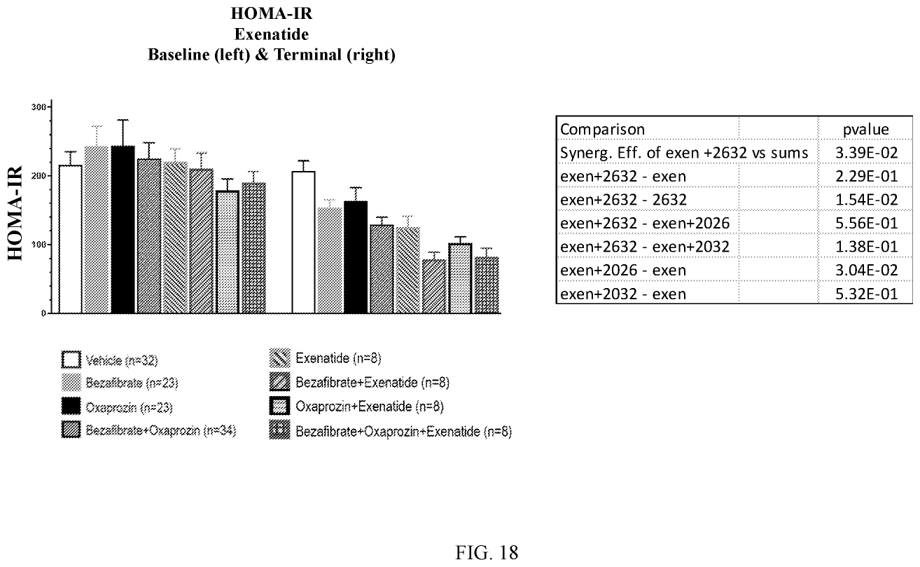

Bezafibrate (60 mg/kg)+oxaprozin (50 mg/kg) over 34 days, compared to treatment with vehicle, induced significant decreases in body weight of 11.2% (FIG. 14, pvalue 9.9e-32), body fat mass (FIG. 15, pvalue 1.5e-07), plasma leptin (FIG. 17, pvalue 3.5e-14), plasma insulin (FIG. 6, 4.24e-05) and insulin resistance index (HOMA-IR) (FIG. 18, pvalue 1.2e-05).

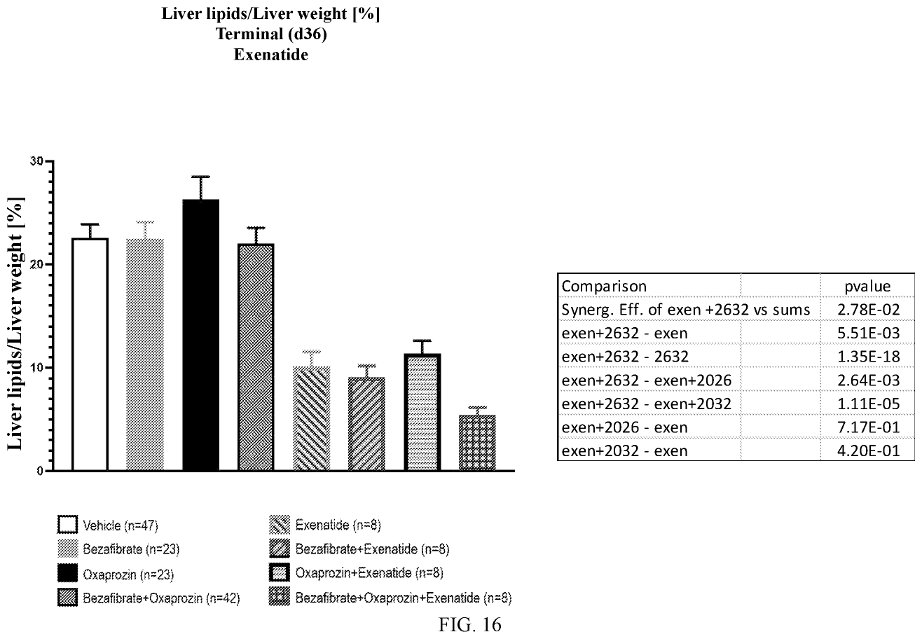

In addition, the combination of bezafibrate (60 mg/kg)+oxaprozin (50 mg/kg) with exenatide (0.05 mg/kg) caused further decreases beyond exenatide alone in several of these metabolic parameters. Surprisingly, the weight loss-inducing effect of the combination with exenatide (FIG. 14) as well as the effects on body fat (FIG. 15) and hepatic fat (FIG. 16) were greater than additive. In fact, bezafibrate+oxaprozin+exenatide produced highly significant reduction in body weight in DIO mice over 34 days (p<0.0001). For body weight, we evaluate drug effects based on the percent change (on day 35). The observed synergistic effect was an additional 14.1 percent of reduction in body weight beyond the sum of the EGS2632 and exenatide groups as a result of concurrent treatment with EGS2632 with exenatide (pvalue 2.19e-09).

Comparing the combination of bezafibrate (60 mg/kg)+oxaprozin (50 mg/kg) with exenatide to the sum of the bezafibrate/ozaprozin and exenatide alone groups showed an additional reduction in liver fat as a result of treatment with bezafibrate/ozaprozin with exenatide (pvalue 2.78e-02); there is furthermore an additional reduction in body fat as a result of treatment with EGS2632 with exenatide (pvalue 4.77e-02).

Bezafibrate+oxaprozin with exenatide lowered leptin levels beyond that achieved with exenatide alone (pvalue 8.5e-03). Bezafibrate+oxaprozin with exenatide synergistically reduced HOMA-IR by 21.9 percent greater than the sum of the bezafibrate+oxaprozin and exenatide groups (pvalue 3.4e-02).

In summary, the magnitude of the effects of the combination of bezafibrate+oxaprozin and exenatide on body weight and other metabolic parameters could not have been anticipated based on the known effects of these individual agents. In fact, surprisingly, synergistic effects were observed on several parameters of metabolic status.

### Example 4: The Combination of Bezafibrate and Oxaprozin Plus Lixisenatide Induces Greater than Expected Body Weight Loss, Body Fat Loss, and Reduction in Hepatosteatosis in a Mouse Model of Obesity and Pre-Diabetes

Applicants previously demonstrated that agents which enhance energy expenditure, such as those promoting the differentiation of human or non-human brown adipocyte progenitor cells into brown adipocytes, i.e., an agent that recruits brown adipocytes or BAT in vivo, can cause improvement in parameters of metabolic health in obese individuals or animals or diabetic individuals or animals or individuals of animals with other metabolic conditions, such as decreases in body weight, body fat content, plasma levels of leptin, glucose, insulin, and an index of insulin resistance, the Homeostatic Model Assessment for Insulin Resistance (HOMA-IR). The HOMA-IR equals the plasma insulin [microIU/ml]×plasma glucose [mM])/22.5).

These findings were made in the commonly used mouse model of environmental obesity and pre-diabetes, or insulin resistance, the Diet-Induced Obese (DIO) mouse. In order to uncover possible greater effects between agents that increase energy expenditure, for example by recruiting brown adipocytes, and an agent that decreases body weight by reducing food intake, we investigated the effects of the combination of bezafibrate and oxaprozin and the GLP-1 receptor agonist lixisenatide.

Bezafibrate and oxaprozin both recruit brown adipocytes and are known, or are believed, to affect different molecular targets and intracellular signaling pathways. Lixisenatide decreases food intake though activation of the GLP-1 receptor.

### Materials and Methods

### Animal Studies

Obesity and insulin resistance, an early stage in the development of type 2 diabetes (also known as pre-diabetes), was induced in C57Bl/6 mice by feeding the mice with a high fat diet (Research Diets, Cat #D12492, 60% fat kcal) for 12 weeks starting at 6 weeks of age, and throughout the period of compound dosing. The mice were housed at 22-23° C. and abundant nestlets/bedding material was provided to allow the animals to maintain a microenvironment close to their thermoneutrality of 28-30° C., starting 2 weeks prior to the dosing period and for the full dosing period with a 12 h/12 h light/dark cycle. These environmental conditions are understood by those skilled in the art to reduce the stimulus for maintaining BAT, a thermogenic tissue that is recruited physiologically by cold stimulus, and permit a wider window for observing effects related to BAT recruitment.

Mice were dosed once per day by oral gavage (100 μl per mouse) with vehicle (PBS+0.5% CMC+0.1% Tween-80) alone or with bezafibrate (60 mg/kg)+oxaprozin (50 mg/kg) (also referred to as EGS2632) dissolved in the vehicle, for 34 days. In addition, the mice received every day by intraperitoneal injection (100 μl per mouse) either vehicle (9.6 mg/ml mannitol, 4.8 mg/ml sucrose, 0.37 mg/ml L-histidine, 0.025 mg/ml polysorbate 20 (Tween 20, CAS #9005-64-5), pH adjusted to 7.4 with HCl) or lixisenatide (0.243 mg/kg) dissolved in the vehicle.

Body weight was recorded every day, body composition (fat and lean mass with EchoMRI) was assessed at the end of the study (University of Cincinnati Mouse Metabolic Phenotyping Center). At the end of the dosing period, animals were euthanized by CO₂, and a piece (ca. 50 mg) of liver was collected and frozen for triglyceride quantification (Triglyceride Quantification Kit, Sigma-Aldrich, St. Louis, MO). Blood plasma was isolated from submandibular blood collected from animals fasted for 6 hours at baseline and at the end of the dosing period. Plasma glucose, insulin and leptin levels were assessed at baseline and at the end of the dosing period (University of Cincinnati Mouse Metabolic Phenotyping Center). Insulin sensitivity was determined using the homeostasis model assessment of insulin resistance (HOMA-IR).

### Statistical Analysis

Data from in vivo mouse studies are presented as means±SEM. Significance values were evaluated based on the Z-test with normal approximations. For body fat and liver fat, since the distributions of these values were both skewed, we used the log-transformed values to better approximate the normal distribution. To assess synergistic effects of A and B, the combination A+B was compared to the sum of A alone and B alone. To quantify synergistic effects, we used percent changes from baseline when baseline data were available and used the difference from vehicle for parameters without baseline data.

### Results

Applicants tested the effects of bezafibrate (60 mg/kg)+oxaprozin (50 mg/kg), lixisenatide (0.243 mg/kg) alone, and the combination of bezafibrate (60 mg/kg)+oxaprozin (50 mg/kg) with lixisenatide (0.243 mg/kg) in DIO mice over 34 days of dosing on parameters of metabolic health.

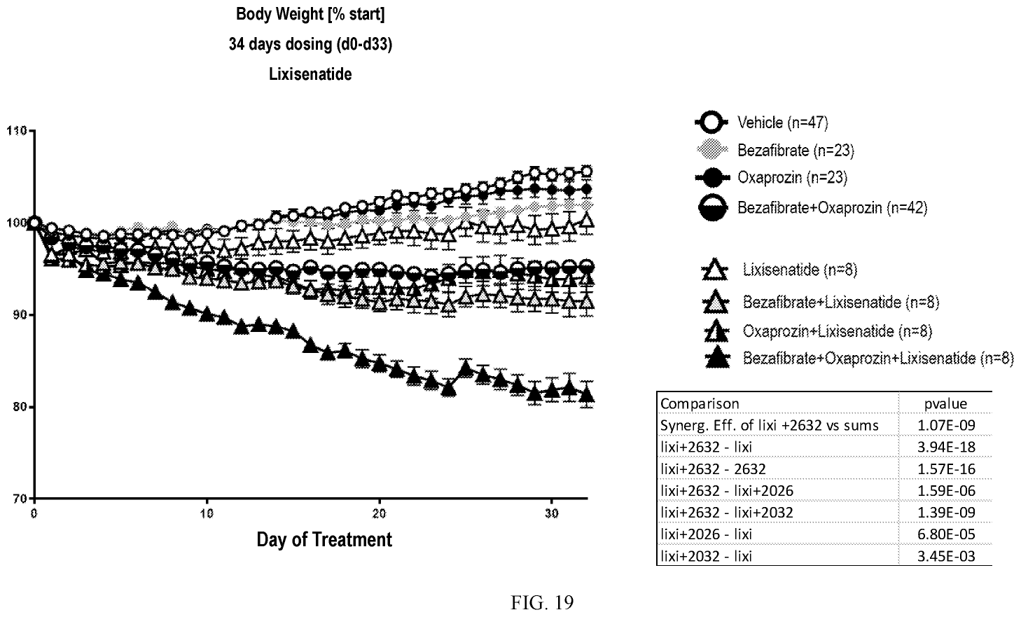

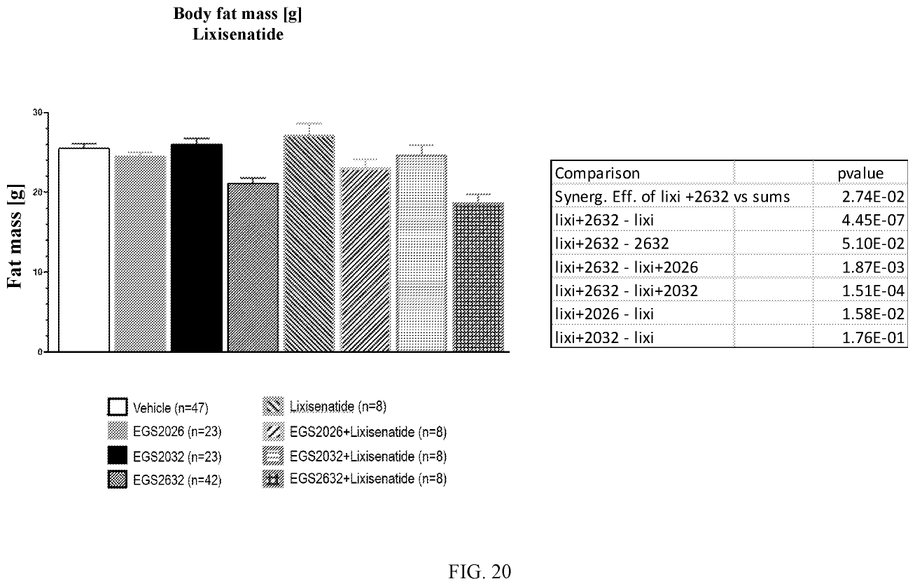

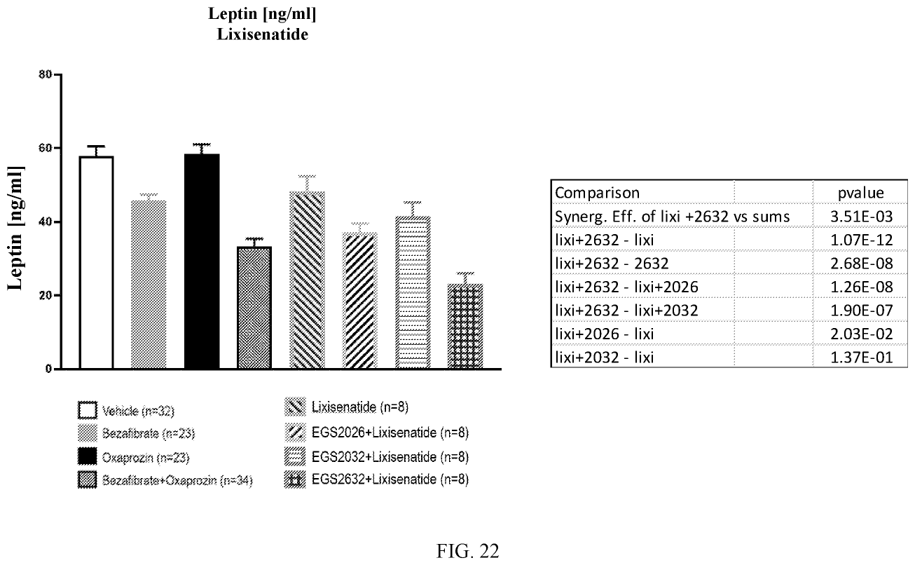

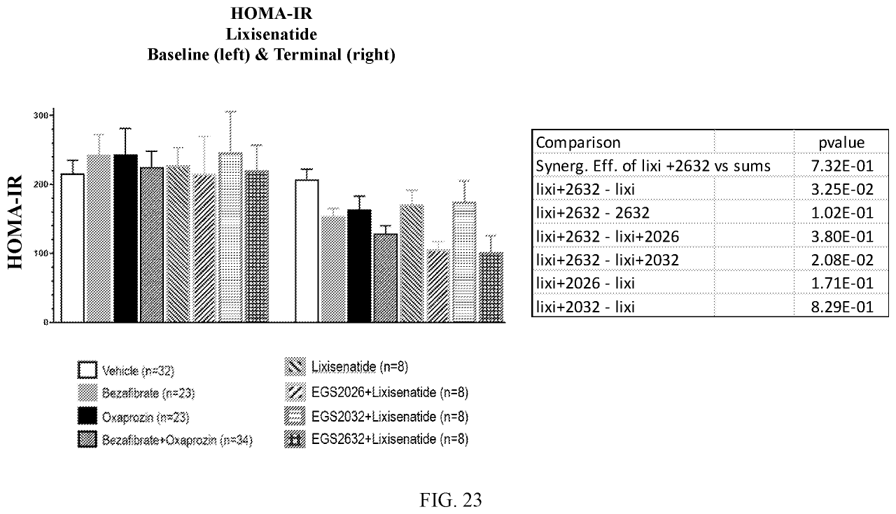

Bezafibrate (60 mg/kg)+oxaprozin (50 mg/kg) over 34 days, compared to treatment with vehicle, induced significant decreases in body weight of 11.2% (FIG. 19, pvalue 9.9e-32), body fat mass (FIG. 20, pvalue 1.5e-07), plasma leptin (FIG. 22, pvalue 3.5e-14), plasma insulin (FIG. 6, 4.24e-05) and insulin resistance index (HOMA-IR) (FIG. 23, pvalue 1.2e-05).

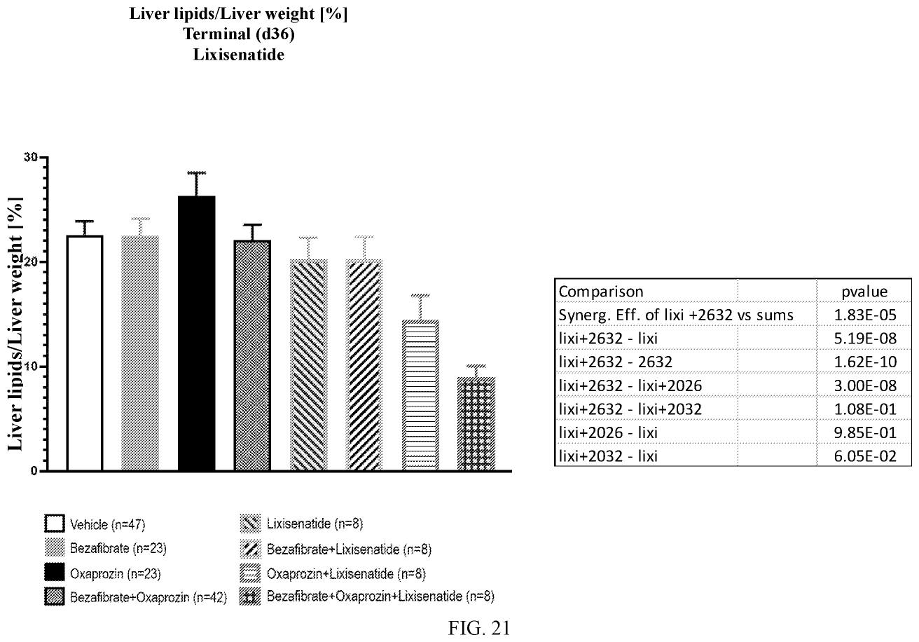

In addition, the combination of bezafibrate (60 mg/kg)+oxaprozin (50 mg/kg) with lixisenatide (0.243 mg/kg) caused further decreases beyond lixisenatide alone in several of these metabolic parameters. Surprisingly, the weight loss-inducing effect of the combination with lixisenatide (FIG. 19) as well as the effects on body fat (FIG. 20) and liver fat (FIG. 21) were greater than additive. In fact, bezafibrate+oxaprozin+lixisenatide produced highly significant reduction in body weight in DIO mice over 34 days (p<0.0001). The observed synergistic effect was an additional 13.7 percent reduction in body weight beyond the sum of the bezafibrate+oxaprozin and lixisenatide groups as a result of concurrent treatment with bezafibrate+oxaprozin with lixisenatide (pvalue 1.07e-09).

Comparing the combination of bezafibrate (60 mg/kg)+oxaprozin (50 mg/kg) with lixisenatide to the sum of the bezafibrate/ozaprozin and lixisenatide alone groups showed an additional reduction in liver fat as a result of treatment with bezafibrate/ozaprozin with lixisenatide (pvalue 1.83e-05); there is furthermore an additional reduction in body fat as a result of treatment with EGS2632 with lixisenatide (pvalue 2.74e-02).

Bezafibrate+oxaprozin with lixisenatide synergistically lowered leptin levels from baseline by 22.7 percent beyond the sum of the bezafibrate/ozaprozin and lixisenatide alone groups (pvalue 3.51e-03). Bezafibrate+oxaprozin with lixisenatide reduced HOMA-IR to a level greater than lixisenatide alone (pvalue 3.25e-02).

In summary, the magnitude of the effects of the combination of bezafibrate+oxaprozin and lixisenatide on body weight and other metabolic parameters could not have been anticipated based on the known effects of these individual agents. In fact, surprisingly, synergistic effects were observed on several parameters of metabolic status.

### Example 5: The Combination of Bezafibrate and Oxaprozin Plus Dulaglutide Induces Greater than Expected Body Weight Loss, Body Fat Loss, and Reduction in Hepatosteatosis in a Mouse Model of Obesity and Pre-Diabetes

Applicants previously demonstrated that agents which enhance energy expenditure, such as those promoting the differentiation of human or non-human brown adipocyte progenitor cells into brown adipocytes, i.e., an agent that recruits brown adipocytes or BAT in vivo, can cause improvement in parameters of metabolic health in obese individuals or animals or diabetic individuals or animals or individuals of animals with other metabolic conditions, such as decreases in body weight, body fat content, plasma levels of leptin, glucose, insulin, and an index of insulin resistance, the Homeostatic Model Assessment for Insulin Resistance (HOMA-IR). The HOMA-IR equals the plasma insulin [microIU/ml]×plasma glucose [mM])/22.5).

These findings were made in the commonly used mouse model of environmental obesity and pre-diabetes, or insulin resistance, the Diet-Induced Obese (DIO) mouse. In order to uncover possible greater effects between agents that increase energy expenditure, for example by recruiting brown adipocytes, and an agent that decreases body weight by reducing food intake, we investigated the effects of the combination of bezafibrate and oxaprozin and the GLP-1 receptor agonist dulaglutide.

Bezafibrate and oxaprozin both recruit brown adipocytes and are known, or are believed, to affect different molecular targets and intracellular signaling pathways. Dulaglutide decreases food intake though activation of the GLP-1 receptor.

### Materials and Methods

### Animal Studies

Obesity and insulin resistance, an early stage in the development of type 2 diabetes (also known as pre-diabetes), was induced in C57Bl/6 mice by feeding the mice with a high fat diet (Research Diets, Cat #D12492, 60% fat kcal) for 12 weeks starting at 6 weeks of age, and throughout the period of compound dosing. The mice were housed at 22-23° C. and abundant nestlets/bedding material was provided to allow the animals to maintain a microenvironment close to their thermoneutrality of 28-30° C., starting 2 weeks prior to the dosing period and for the full dosing period with a 12 h/12 h light/dark cycle. These environmental conditions are understood by those skilled in the art to reduce the stimulus for maintaining BAT, a thermogenic tissue that is recruited physiologically by cold stimulus, and permit a wider window for observing effects related to BAT recruitment.

Mice were dosed once per day by oral gavage (100 μl per mouse) with vehicle (PBS+0.5% CMC+0.1% Tween-80) alone or with bezafibrate (60 mg/kg)+oxaprozin (50 mg/kg) (also referred to as EGS2632) dissolved in the vehicle, for 34 days. In addition, the mice received every 7 days by intraperitoneal injection (100 μl per mouse) either vehicle (9.6 mg/ml mannitol, 4.8 mg/ml sucrose, 0.37 mg/ml L-histidine, 0.025 mg/ml polysorbate 20 (Tween 20, CAS #9005-64-5), pH adjusted to 7.4 with HCl) or dulaglutide (0.6 mg/kg) dissolved in the vehicle.

Body weight was recorded every day, body composition (fat and lean mass with EchoMRI) was assessed at the end of the study (University of Cincinnati Mouse Metabolic Phenotyping Center). At the end of the dosing period, animals were euthanized by CO₂, and a piece (ca. 50 mg) of liver was collected and frozen for triglyceride quantification (Triglyceride Quantification Kit, Sigma-Aldrich, St. Louis, MO). Blood plasma was isolated from submandibular blood collected from animals fasted for 6 hours at baseline and at the end of the dosing period. Plasma glucose, insulin and leptin levels were assessed at baseline and at the end of the dosing period (University of Cincinnati Mouse Metabolic Phenotyping Center). Insulin sensitivity was determined using the homeostasis model assessment of insulin resistance (HOMA-IR).

### Statistical Analysis

Data from in vivo mouse studies are presented as means±SEM. Significance values were evaluated based on the Z-test with normal approximations. For body fat and liver fat, since the distributions of these values were both skewed, we used the log-transformed values to better approximate the normal distribution. To assess synergistic effects of A and B, the combination A+B was compared to the sum of A alone and B alone. To quantify synergistic effects, we used percent changes from baseline when baseline data were available and used the difference from vehicle for parameters without baseline data.

### Results

Applicants tested the effects of bezafibrate (60 mg/kg)+oxaprozin (50 mg/kg), dulaglutide (0.6 mg/kg) alone, and the combination of bezafibrate (60 mg/kg)+oxaprozin (50 mg/kg) with dulaglutide (0.6 mg/kg) in DIO mice over 34 days of dosing on parameters of metabolic health.

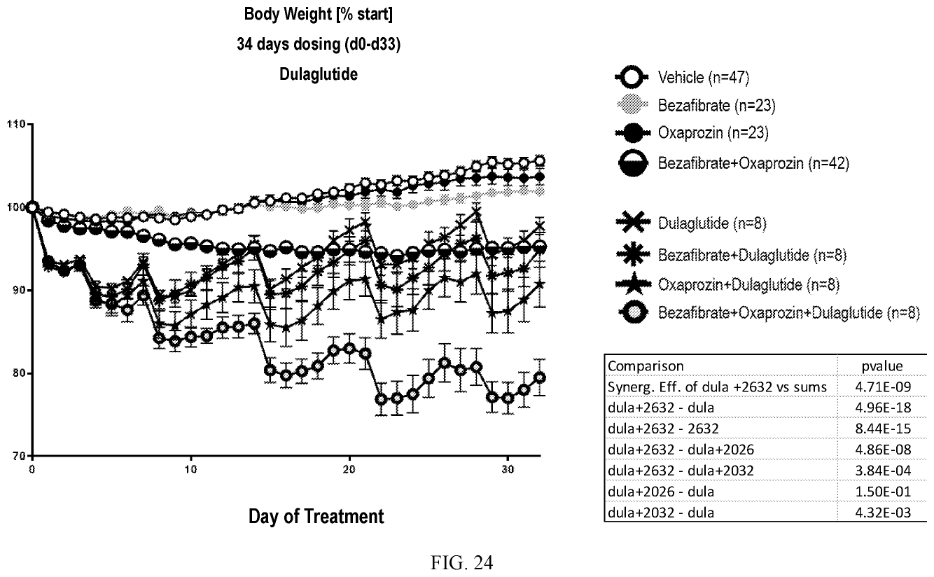

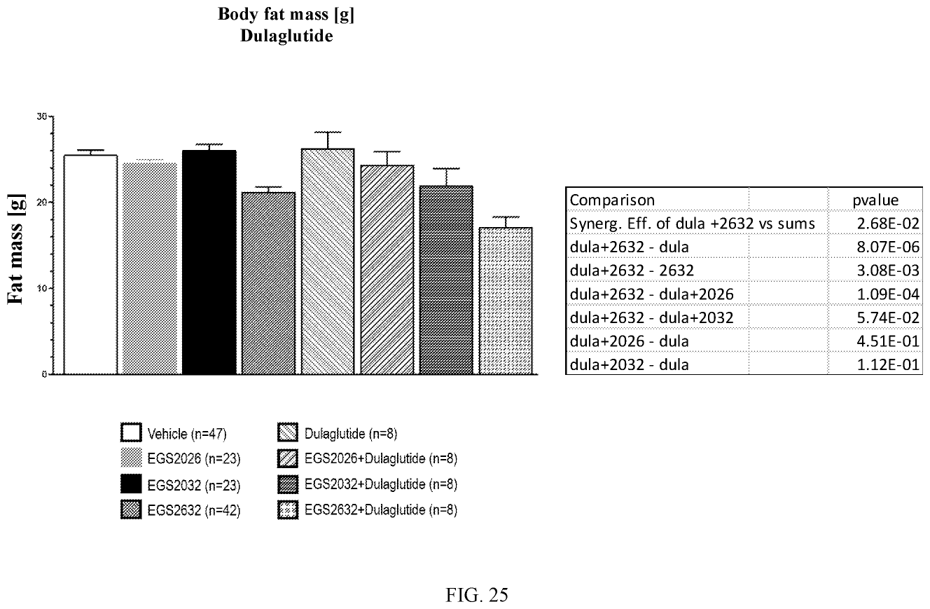

Bezafibrate (60 mg/kg)+oxaprozin (50 mg/kg) over 34 days, compared to treatment with vehicle, induced significant decreases in body weight of 11.2% (FIG. 24, pvalue 9.9e-32), body fat mass (FIG. 25, pvalue 1.5e-07), plasma leptin (FIG. 22, pvalue 3.5e-14), plasma insulin (FIG. 6, 4.24e-05) and insulin resistance index (HOMA-IR) (FIG. 23, pvalue 1.2e-05).

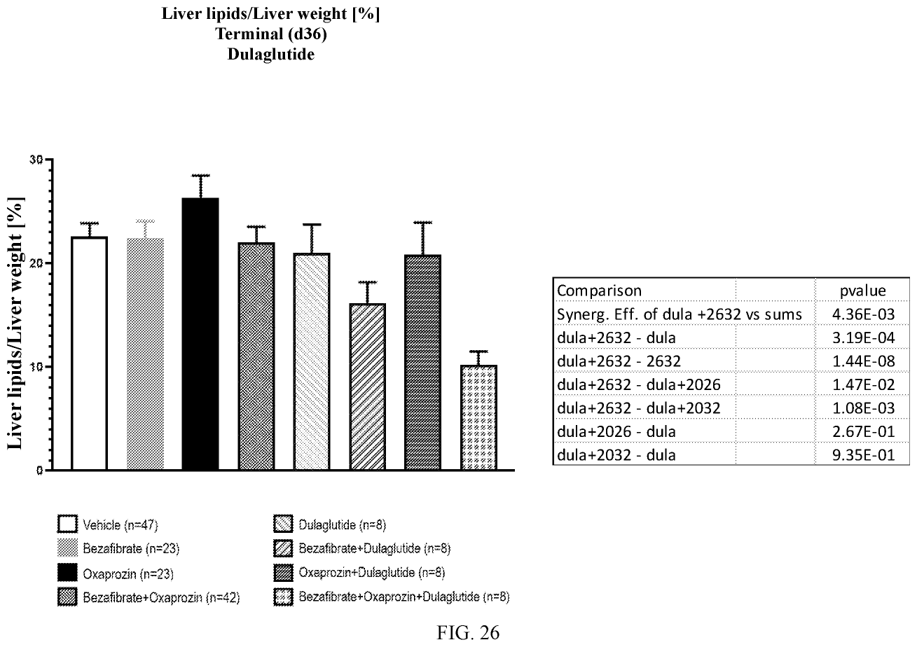

In addition, the combination of bezafibrate (60 mg/kg)+oxaprozin (50 mg/kg) with dulaglutide (0.6 mg/kg) caused further decreases beyond dulaglutide alone in several of these metabolic parameters. Surprisingly, the weight loss-inducing effect of the combination with dulaglutide (FIG. 24) as well as the effects on body fat (FIG. 25) and liver fat (FIG. 26) were greater than additive. In fact, bezafibrate+oxaprozin+dulaglutide produced highly significant reduction in body weight in DIO mice over 34 days (p<0.0001). For body weight, we evaluated drug effects based on the percent change from baseline on day 35 (generally), however in the case of dulaglutide, which was dosed every 7 days, we used the average weights on days 29-35. The observed synergistic effect was an additional 13.6 percent reduction in body weight beyond the sum of the bezafibrate+oxaprozin and dulaglutide groups as a result of concurrent treatment with bezafibrate+oxaprozin with dulaglutide (pvalue 4.71-09).

Comparing the combination of bezafibrate (60 mg/kg)+oxaprozin (50 mg/kg) with dulaglutide to the sum of the bezafibrate/ozaprozin and dulaglutide alone groups showed an additional reduction in liver fat as a result of treatment with bezafibrate/ozaprozin with dulaglutide (pvalue 4.36e-03); there is furthermore an additional reduction in body fat as a result of treatment with EGS2632 with dulaglutide (pvalue 2.68e-02).

In summary, the magnitude of the effects of the combination of bezafibrate+oxaprozin and dulaglutide on body weight and other metabolic parameters could not have been anticipated based on the known effects of these individual agents. In fact, surprisingly, synergistic effects were observed on several parameters of metabolic status.

Given these surprising yet conclusive data generated with 4 different marketed GLP-1 agonists showing synergistic effects between EGS2632 and each individual GLP-1 agonist drug on body weight, body fat, and liver fat, as well as improvements beyond the individual GLP-1 agonist drugs (additive and even synergistic effects in some cases) on leptin and HOMA-IR, it can be concluded that these are general effects of EGS2632 when administered with any drug comprising GLP-1 agonism as its mechanism, either in whole or in part.

SEQ ID NO 1:
SYDYMEGGDIRVRRLFCRTQWYLRIDKRGKVKGTQEMKNNYNIMEIR
TVAVGIVAIKGVESEFYLAMNKEGKLYAKKECNEDCNFKELILENHY
NTYASAKWTHNGGEMFVALNQKGIPVRGKKTKKEQKTAHFLPMAIT
SEQ ID NO 2:
CNDMTPEQMATNVNCSSPERHTRSYDYMEGGDIRVRRLFCRTQWYLR
IDKRGKVKGTQEMKNNYNIMEIRTVAVGIVAIKGVESEFYLAMNKEG
KLYAKKECNEDCNFKELILENHYNTYASAKWTHNGGEMFVALNQKGI
PVRGKKTKKEQKTAHFLPMAITAEPKSSDKTHTCPPCPAPELLGGPS
VFLFPPKPKDTLMISRTPEVTCVVVDVSHEDPEVKFNWYVDGVEVHN
AKTKPREEQYASTYRVVSVLTVLHQDWLNGKEYKCKVSNKALPAPIE
KTISKAKGQPREPQVYTLPPSRDELTKNQVSLTCLVKGFYPSDIAVE
WESNGQPENNYKTTPPVLDSDGSFFLYSKLTVDKSRWQQGNVFSCSV
MHEALHNHYTQKSLSLSPGK
SEQ ID NO 3:
APLEPKSSDKTHTCPPCPAPELLGGPSVFLFPPKPKDTLMISRTPEV
TCVVVDVSHEDPEVKFNWYVDGVEVHNAKTKPREEQYASTYRVVSVL
TVLHQDWLNGKEYKCKVSNKALPAPIEKTISKAKGQPREPQVYTLPP
SRDELTKNQVSLTCLVKGFYPSDIAVEWESNGQPENNYKTTPPVLDS
DGSFFLYSKLTVDKSRWQQGNVFSCSVMHEALHNHYTQKSLSLSPGG
GGSGGGGSCNDMTPEQMATNVNCSSPERHTRSYDYMEGGDIRVRRLF
CRTQWYLRIDKRGKVKGTQEMKNNYNIMEIRTVAVGIVAIKGVESEF
YLAMNKEGKLYAKKECNEDCNFKELILENHYNTYASAKWTHNGGEMF
VALNQKGIPVRGKKTKKEQKTAHFLPMAIT
SEQ ID NO 4:
CNDMTPEQMATNVNCSSPERHTRSYDYMEGGDIRVRRLFCRTQWYLR
IDKRGKVKGTQEMKNNYNIMEIRTVAVGIVAIKGVESEFYLAMNKEG
KLYAKKECNEDCNFKELILENHYNTYASAKWTHNGGEMFVALNQKGI
PVRGKKTKKEQKTAHFLPMAITERKSSVECPPCPAPPVAGPSVFLFP
PKPKDTLMISRTPEVTCVVVDVSHEDPEVQFNWYVDGVEVHNAKTKP
REEQFNSTFRVVSVLTVVHQDWLNGKEYKCKVSNKGLPAPIEKTISK
TKGQPREPQVYTLPPSREEMTKNQVSLTCLVKGFYPSDISVEWESNG
QPENNYKTTPPMLDSDGSFFLYSKLTVDKSRWQQGNVFSCSVMHEAL
HNHYTQKSLSLSPGK
SEQ ID NO 5:
APLERKSSVECPPCPAPPVAGPSVFLFPPKPKDTLMISRTPEVTCVV
VDVSHEDPEVQFNWYVDGVEVHNAKTKPREEQFNSTFRVVSVLTVVH
QDWLNGKEYKCKVSNKGLPAPIEKTISKTKGQPREPQVYTLPPSREE
MTKNQVSLTCLVKGFYPSDISVEWESNGQPENNYKTTPPMLDSDGSF
FLYSKLTVDKSRWQQGNVFSCSVMHEALHNHYTQKSLSLSPGKGGGG
SGGGGSCNDMTPEQMATNVNCSSPERHTRSYDYMEGGDIRVRRLFCR
TQWYLRIDKRGKVKGTQEMKNNYNIMEIRTVAVGIVAIKGVESEFYL
AMNKEGKLYAKKECNEDCNFKELILENHYNTYASAKWTHNGGEMFVA
LNQKGIPVRGKKTKKEQKTAHFLPMAIT
SEQ ID NO 6:
CNDMTPEQMATNVNCSSPERHTRSYDYMEGGDIRVRRLFCRTQWYLR
IDKRGKVKGTQEMKNNYNIMEIRTVAVGIVAIKGVESEFYLAMNKEG
KLYAKKECNEDCNFKELILENHYNTYASAKWTHNGGEMFVALNQKGI
PVRGKKTKKEQKTAHFLPMAITESKYGPPCPPCPAPEFLGGPSVFLF
PPKPKDTLMISRTPEVTCVVVDVSQEDPEVQFNWYVDGVEVHNAKTK
PREEQFNSTYRVVSVLTVLHQDWLNGKEYKCKVSNKGLPSSIEKTIS
KAKGQPREPQVYTLPPSQEEMTKNQVSLTCLVKGFYPSDIAVEWESN
GQPENNYKTTPPVLDSDGSFFLYSRLTVDKSRWQEGNVFSCSVMHEA
LHNHYTQKSLSLSLG
SEQ ID NO 7:
CNDMTPEQMATNVNCSSPERHTRSYDYMEGGDIRVRRLFCRTQWYLR
IDKRGKVKGTQEMKNNYNIMEIRTVAVGIVAIKGVESEFYLAMNKEG
KLYAKKECNEDCNFKELILENHYNTYASAKWTHNGGEMFVALNQKGI
PVRGKKTKKEQKTAHFLPMAITGGGGSESKYGPPCPPCPAPEFLGGP
SVFLFPPKPKDTLMISRTPEVTCVVVDVSQEDPEVQFNWYVDGVEVH
NAKTKPREEQFNSTYRVVSVLTVLHQDWLNGKEYKCKVSNKGLPSSI
EKTISKAKGQPREPQVYTLPPSQEEMTKNQVSLTCLVKGFYPSDIAV
EWESNGQPENNYKTTPPVLDSDGSFFLYSRLTVDKSRWQEGNVFSCS
VMHEALHNHYTQKSLSLSLG
SEQ ID NO 8:
MHKWILTWILPTLLYRSCFHIICLVGTISLACNDMTPEQMATNVNCS
SPERHTRSYDYMEGGDIRVRRLFCRTQWYLRIDKRGKVKGTQEMKNN
YNIMEIRTVAVGIVAIKGVESEFYLAMNKEGKLYAKKECNEDCNFKE
LILENHYNTYASAKWTHNGGEMFVALNQKGIPVRGKKTKKEQKTAHF
LPMAIT

1-27. (canceled)

28. A pharmaceutical composition comprising:
a) bezafibrate as a first active ingredient, oxaprozin as a second active ingredient, and a glucagon-like peptide-1 (GLP-1) receptor agonist as a third active ingredient and a pharmaceutically acceptable carrier; or
b) a GLP-1 receptor agonist as a first active ingredient, fibroblast growth factor 7 (FGF7) or analogs thereof as a second active ingredient and a pharmaceutically acceptable carrier.

29. The pharmaceutical composition of claim 28, comprising a GLP-1 receptor agonist, bezafibrate and oxaprozin.

30. The pharmaceutical composition of claim 29, comprising: (a) a therapeutically effective amount of bezafibrate ranging from about 25% to about 75% of the clinically approved dosage of BEZALIP® SR (bezafibrate sustained release); and (b) a therapeutically effective amount of oxaprozin ranging from about 25% to about 100% of the clinically approved dosage of DAYPRO® (oxaprozin).

31. The pharmaceutical composition of claim 30, wherein the therapeutically effective amount of bezafibrate ranges from about 100 mg to about 400 mg, and wherein the therapeutically effective amount of oxaprozin range from about 200 mg to about 2000 mg.

32. The pharmaceutical composition of claim 29, comprising: (a) a therapeutically effective amount of bezafibrate ranging from about 25% to about 100% of the clinically approved dosage of BEZALIP® SR; and (b) a therapeutically effective amount of oxaprozin ranging from about 25% to about 75% of the clinically approved dosage of DAYPRO®.

33. The pharmaceutical composition of claim 32, wherein the therapeutically effective amount of bezafibrate ranges from about 100 mg to about 400 mg or about 5 mg to about 500 mg, and wherein the therapeutically effective amount of oxaprozin ranges from about 200 mg to about 1800 mg or about 250 mg to about 500 mg.

34. The pharmaceutical composition of claim 28, wherein the therapeutically effective amount of said GLP-1 receptor agonist is about 10% to about 100% of the clinically approved dosage of said GLP-1 receptor agonist.

35. The pharmaceutical composition of claim 28, comprising a GLP-1 receptor agonist and FGF-7.

36. The pharmaceutical composition of claim 35, wherein the therapeutically effective amount of said GLP-1 receptor agonist is about 10% to about 100% of the clinically approved dosage of said GLP-1 receptor agonist and the FGF-7 dosage is about 0.02 to about 0.1 mg/kg, about 0.04 to about 0.08 mg/kg or about 10% to about 100% of said FGF-7 dosage.

37. The pharmaceutical composition of claim 28, wherein the GLP-1 receptor agonist is selected from the group consisting of: dulaglutide, semaglutide, exenatide, liraglutide, lixisenatide, albiglutide, tirzepatide, danuglipron (PF-06882961), PF-07081532, LY3502970, and combinations thereof.

38. The pharmaceutical composition of claim 28, wherein the composition is in an injectable form or a solid dosage form.

39. The pharmaceutical composition of claim 28, wherein said composition has one or more biological activities selected from the group consisting of:
(a) an increase in thermogenesis in brown adipose tissue, skeletal muscle tissue and/or white adipose tissue; (b) an increase in insulin sensitivity of skeletal muscle, white adipose tissue, or liver; (c) an increase in glucose tolerance; (d) an increase in basal respiration, maximal respiration rate, or uncoupled respiration; (e) an increase in metabolic rate; (f) a decrease in hepatosteatosis; (g) a decrease in body weight; (h) a decrease in body fat mass; (i) a decrease in plasma leptin levels; (j) a decrease in glycemia; (k) a decrease in plasma insulin levels; (1) a decrease in insulin resistance; (m) a decrease in circulating lipid levels; or a combination thereof.

40. The pharmaceutical composition of claim 39, wherein the metabolic disorder is one or more of obesity, overweight, type II diabetes, insulin resistance, hyperinsulinemia, hyperglycemia, pre-diabetes, hypertension, hyperlipidemia, hepatosteatosis, fatty liver, non-alcoholic fatty liver disease, hyperuricemia, polycystic ovarian syndrome, acanthosis nigricans, hyperphagia, endocrine abnormalities, triglyceride storage disease, Bardet-Biedl syndrome, Laurence-Moon syndrome, Prader-Willi syndrome, neurodegenerative diseases, and Alzheimer's disease.

41. The pharmaceutical composition of claim 28, wherein the therapeutically effective amount of said GLP-1 receptor agonist is about 10% to about 100% of the clinically approved dosage of said GLP-1 receptor agonist.

42. The pharmaceutical composition of claim 28, wherein the FGF7 or analog thereof comprises SEQ ID NO: 1, 2, 3, 4, 5, 6, 7, or 8.

43. A method of promoting brown adipogenesis in a subject in need thereof, the method comprising administering to the subject the pharmaceutical composition of claim 28.

44. The method of claim 43, further comprising modulating a metabolic response in the subject and/or treating a metabolic disorder in the subject.

45. The method of claim 44, wherein the metabolic disorder is one or more of obesity, overweight, type II diabetes, insulin resistance, hyperinsulinemia, hyperglycemia, pre-diabetes, hypertension, hyperlipidemia, hepatosteatosis, fatty liver, non-alcoholic fatty liver disease, hyperuricemia, polycystic ovarian syndrome, acanthosis nigricans, hyperphagia, endocrine abnormalities, triglyceride storage disease, Bardet-Biedl syndrome, Laurence-Moon syndrome, Prader-Willi syndrome, neurodegenerative diseases, and Alzheimer's disease.

46. The method of claim 43, wherein the FGF7 or analog thereof comprises SEQ ID NO: 1, 2, 3, 4, 5, 6, 7, or 8.

47. A method of promoting brown adipogenesis in a subject in need thereof, the method comprising administering to the subject the pharmaceutical composition of claim 28.

48. The method of claim 47, further comprising modulating a metabolic response in the subject and/or treating a metabolic disorder in the subject.

49. The method of claim 48, wherein the metabolic disorder is one or more of obesity, overweight, type II diabetes, insulin resistance, hyperinsulinemia, hyperglycemia, pre-diabetes, hypertension, hyperlipidemia, hepatosteatosis, fatty liver, non-alcoholic fatty liver disease, hyperuricemia, polycystic ovarian syndrome, acanthosis nigricans, hyperphagia, endocrine abnormalities, triglyceride storage disease, Bardet-Biedl syndrome, Laurence-Moon syndrome, Prader-Willi syndrome, neurodegenerative diseases, and Alzheimer's disease.


SEQUENCE LISTING (8 sequences)

SEQ ID NO: 1 - protein, 140 aa, Artificial Sequence
  analogs of human fibroblast growth factors
SYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 2 - protein, 396 aa, Artificial Sequence
  analogs of human fibroblast growth factors
CNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAITAEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 406 aa, Artificial Sequence
  analogs of human fibroblast growth factors
APLEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSCNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 4 - protein, 391 aa, Artificial Sequence
  analogs of human fibroblast growth factors
CNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAITERKSSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 5 - protein, 404 aa, Artificial Sequence
  analogs of human fibroblast growth factors
APLERKSSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGKGGGGSGGGGSCNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 6 - protein, 391 aa, Artificial Sequence
  analogs of human fibroblast growth factors
CNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAITESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 7 - protein, 396 aa, Artificial Sequence
  analogs of human fibroblast growth factors
CNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAITGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 8 - protein, 194 aa, Homo sapiens
  MISC_FEATURE (1)..(194): Fibroblast Growth Factor-7
MHKWILTWILPTLLYRSCFHIICLVGTISLACNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT